(19) United States
(12) Patent Application Publication
(10) Pub. No.: US 2012/0220571 A1
(43) Pub. Date: Aug. 30, 2012

(54) HIV INTEGRASE INHIBITORS

(76) Inventors: John S. Wai, Harleysville, PA (US); Dai-shi Su, Dresher, PA (US); Catherine M. Wiscount, Allentown, PA (US)

(21) Appl. No.: 13/392,169

(22) PCT Filed: Aug. 16, 2010
(86) PCT No.: PCT/US2010/045589

§ 371 Date: May 9, 2012

Related U.S. Application Data
(60) Provisional application No. 61/236,987, filed on Aug. 26, 2009.

(51) Int. Cl. A61K 31/436; C07D 471/04; A61P 37/00; C07D 487/14; A61K 31/55; A61P 31/18; C07D 491/052; A61K 31/4375

(52) U.S. Cl. 514/214.02; 546/116; 514/302; 546/123; 514/300; 540/579

(57) ABSTRACT

Compounds of Formula I are inhibitors of HIV integrase and inhibitors of HIV replication: (I), wherein X is O or N(R3); and R1, R2, R3 and Y are defined herein. The compounds are useful for the prophylaxis or treatment of infection by HIV and the prophylaxis, treatment, or delay in the onset or progression of AIDS. The compounds are employed against HIV infection and AIDS as compounds per se (or as hydrates or solvates thereof) or in the form of pharmaceutically acceptable salts. The compounds and their salts can be employed as ingredients in pharmaceutical compositions, optionally in combination with other antivirals, immunomodulators, antibiotics or vaccines.

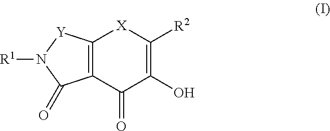

### CROSS-REFERENCE TO RELATED APPLICATIONS

This application claims the benefit of U.S. Provisional Application No. 61/236,987 (filed Aug. 26, 2009), the disclosure of which is hereby incorporated by reference in its entirety.

### FIELD OF THE INVENTION

The present invention is directed to certain 4-pyridinone and 4-pyranone compounds (including hydrates and solvates thereof) and pharmaceutically acceptable salts thereof, their synthesis, and their use as inhibitors of the HIV integrase enzyme. The compounds and hydrates, solvates and pharmaceutically acceptable salts thereof of the present invention are useful for preventing or treating infection by HIV and for preventing or treating or delaying the onset or progression of AIDS.

### BACKGROUND OF THE INVENTION

A retrovirus designated human immunodeficiency virus (HIV), particularly the strains known as HIV type-1 (HIV-1) virus and type-2 (HIV-2) virus, is the etiological agent of the complex disease that includes progressive destruction of the immune system (acquired immune deficiency syndrome; AIDS) and degeneration of the central and peripheral nervous system. This virus was previously known as LAV, HTLV-III, or ARV. A common feature of retrovirus replication is the insertion by virally-encoded integrase of +proviral DNA into the host cell genome, a required step in HIV replication in human T-lymphoid and monocytoid cells. Integration is believed to be mediated by integrase in three steps: assembly of a stable nucleoprotein complex with viral DNA sequences; cleavage of two nucleotides from the 3′ termini of the linear proviral DNA; covalent joining of the recessed 3′ OH termini of the proviral DNA at a staggered cut made at the host target site. The fourth step in the process, repair synthesis of the resultant gap, may be accomplished by cellular enzymes.

Nucleotide sequencing of HIV shows the presence of a poi gene in one open reading frame [Ratner, L. et al., Nature, 313, 277 (1985)]. Amino acid sequence homology provides evidence that the pot sequence encodes reverse transcriptase, integrase and an HIV protease [Toh, H. et al., EMBO J. 4, 1267 (1985); Power, M. D. et al., Science, 231, 1567 (1986); Pearl, L. H. et al., Nature, 329, 351 (1987)]. All three enzymes have been shown to be essential for the replication of HIV.

It is known that some antiviral compounds which act as inhibitors of HIV replication are effective agents in the treatment of AIDS and similar diseases, including reverse transcriptase inhibitors such as azidothymidine (AZT) and efavirenz and protease inhibitors such as indinavir and nelfinavir. The compounds of this invention are inhibitors of HIV integrase and inhibitors of HIV replication. The inhibition of integrase in vitro and HIV replication in cells is a direct result of inhibiting the strand transfer reaction catalyzed by the recombinant integrase in vitro in HIV infected cells.

The following references are of interest as background:
Kinzel et al., Tet. Letters 2007, 48(37): pp. 6552-6555 discloses the synthesis of tetrahydropyridopyrimidones as a scaffold for HIV-1 integrase inhibitors.
Ferrara et al., Tet. Letters 2007, 48(37), pp. 8379-8382 discloses the synthesis of a hexahydropyrimido[1,2-a]azepine-2-carboxamide derivative useful as an HIV integrase inhibitor.
Muraglia et al., J. Med. Chem. 2008, 51: 861-874 discloses the design and synthesis of bicyclic pyrimidinones as potent and orally bioavailable HIV-1 integrase inhibitors.
US2004/229909 discloses certain compounds having integrase inhibitory activity.
U.S. Pat. No. 7,232,819 and US 2007/0083045 disclose certain 5,6-dihydroxypyrimidine-4-carboxamides as HIV integrase inhibitors.
U.S. Pat. No. 7,169,780, U.S. Pat. No. 7,217,713, and US 2007/0123524 disclose certain N-substituted 5-hydroxy-6-oxo-1,6-dihydropyrimidine-4-carboxamides as HIV integrase inhibitors.
U.S. Pat. No. 7,279,487 discloses certain hydroxynaphthyridinone carboxamides that are useful as HIV integrase inhibitors.
U.S. Pat. No. 7,135,467 and U.S. Pat. No. 7,037,908 disclose certain pyrimidine carboxamides that are useful as HIV integrase inhibitors.
U.S. Pat. No. 7,211,572 discloses certain nitrogenous condensed ring compounds that are HIV integrase inhibitors.
U.S. Pat. No. 7,414,045 discloses certain tetrahydro-4H-pyrido[1,2-a]pyrimidine carboxamides, hexahydropyrimido[1,2-a]azepine carboxamides, and related compounds that are useful as HIV integrase inhibitors.
WO 2006/103399 discloses certain tetrahydro-4H-pyrimidooxazepine carboximides, tetrahydropyrazinopyrimidine carboxamides, hexahydropyrimidodiazepine carboxamides, and related compounds that are useful as HIV integrase inhibitors.
US 2007/0142635 discloses processes for preparing hexahydropyrimido[1,2-a]azepine-2-carboxylates and related compounds.
US 2007/0149556 discloses certain hydroxypyrimidinone derivatives having HIV integrase inhibitory activity.
Various pyrimidinone compounds useful as HIV integrase inhibitors are also disclosed in U.S. Pat. No. 7,115,601, U.S. Pat. No. 7,157,447, U.S. Pat. No. 7,173,022, U.S. Pat. No. 7,176,196, U.S. Pat. No. 7,192,948, U.S. Pat. No. 7,273,859, and U.S. Pat. No. 7,419,969.
US 2007/0111984 discloses a series of bicyclic pyrimidinone compounds useful as HIV integrase inhibitors.

### SUMMARY OF THE INVENTION

The present invention is directed to certain 4-pyridinone and 4-pyranone compounds. These compounds (including hydrates and solvates thereof), optionally in the form of pharmaceutically acceptable salts, are useful in the inhibition of retroviral integrases and for the prophylaxis or treatment of infections or other adverse diseases or conditions caused by retroviruses. The compounds of the present invention are, for example, useful in: (a) the inhibition of HIV integrase (e.g., HIV-1 or HIV-2) and SIV, (b) the prophylaxis or treatment of infection by HIV or SIV, and (c) the prophylaxis, treatment, and delay in the onset or progression of AIDS and/or ARC. As another example, the compounds are useful in: (a) the inhibition of XMRV, (b) the prophylaxis or treatment of infection by XMRV and (c) the prophylaxis, treatment or delay in the onset or progression of diseases or conditions caused by XMRV infection such as prostate cancer or chronic fatigue syndrome. In still another example, the compounds are useful in: (a) the inhibition of HTLV (e.g., type 1, type 2 or type 3), (b) the prophylaxis or treatment of infection by HTLV and (c) the prophylaxis, treatment or delay in the onset or progression of diseases or conditions caused by HTLV such as T-cell leukemia or T-cell lymphoma. In the foregoing uses, the compounds can be used per se but are typically employed with one or more other ingredients in pharmaceutical compositions and optionally in combination with other antiretroviral agents, anti-infectives, immunomodulators, antibiotics or vaccines. More particularly, the present invention includes compounds of Formula I and pharmaceutically acceptable salts thereof:

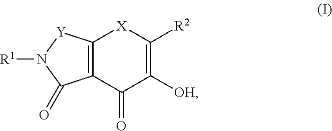

wherein:
X is O or N(R³);
Y is CH(R⁴) or CH(R⁴)CH(R⁴);
R¹ is C₁₋₆ alkyl substituted with R^{J}, wherein R^{J} is AryA or HetA;
R² is H, C₁₋₆ alkyl, or CH(R^{K})R^{L};
R^{K} is H or C₁₋₆ alkyl;
R^{L} is C(R^{T})═CH₂,

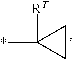

### or C(R^{T})R^{C}—CHR^{C};

R^{T} is H or C₁₋₆ alkyl;
one R^{C} is OH and the other R^{C} is H or OH; or one R^{C} is OH and the other R^{C} is NH₂ or NH—C₁₋₆ alkyl;
R³ is H, C₁₋₆ alkyl, or CH(R^{Q})—C₁₋₆ alkenyl;
R^{Q} is H or C₁₋₆ alkyl;
each R⁴ is independently H or C₁₋₆ alkyl;
alternatively, when X is N(R³), R² and R³ together with the atoms to which they are attached form an azacycloalkyl ring such that the compound of Formula I is a compound of Formula Ia:

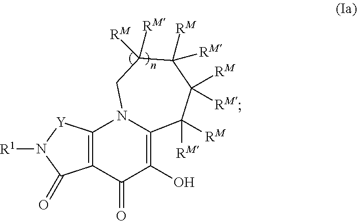

each R^{M} is independently:
(1) H,
(2) C₁₋₆ alkyl,
(3) OH,
(4) C₁₋₆ alkoxy,
(5) oxo which is formed together with the R^{M′} attached to the same carbon, or
(6) methylene which is formed together with an R^{M} on an adjacent ring carbon to provide fused cyclopropyl;
   for each R^{M} which is other than axe, R^{M′} is independently H or C₁₋₆ alkyl;
   n is zero or 1;
   AryA is an aryl which is optionally substituted with a total of from 1 to 5 substituents, wherein:

(i) from zero to 5 substituents are each independently:
(1) C₁₋₆ alkyl,
(2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, N(R^{A})C(O)N(R^{A})R^{B}, or N(R^{A})C(O)C(O)N(R^{A})R^{B},
(3) O—C₁₋₆ alkyl,
(4) C₁₋₆ haloalkyl,
(5) O—C₁₋₆ haloalkyl,
(6) OH,
(7) halogen,
(8) CN,
(9) NO₂,
(10) N(R^{A})R^{B},
(11) C(O)N(R^{A})R^{B},
(12) C(O)R^{A},
(13) C(O)—C₁₋₆ haloalkyl,
(14) C(O)OR^{A},
(15) OC(O)N(R^{A})R^{B},
(16) SR^{A},
(17) S(O)R^{A},
(18) SO₂R^{A},
(19) SO₂N(R^{A})R^{B},
(20) N(R^{A})SO₂R^{B},
(21) N(R^{A})SO₂N(R^{A})R^{B},
(22) N(R^{A})C(O)R^{B},
(23) N(R^{A})C(O)N(R^{A})R^{B},
(24) N(R^{A})C(O)C(O)N(R^{A})R^{B}, or
(25) N(R^{A})CO₂R^{B}, and

(ii) from zero to 2 substituents are each independently:
(1) CycD,
(2) AryD,
(3) HetD,
(4) HetZ,
(5) C₁₋₆ alkyl substituted with CycD, AryD, HetD, or HetZ, or
(6) C(O)-HetZ or C(O)C(O)-HetZ;
   HetA is a heteroaryl which is optionally substituted with a total of from 1 to 5 substituents, wherein:

(i) from zero to 5 substituents are each independently:
(1) C₁₋₆ alkyl,
(2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, N(R^{A})C(O)N(R^{A})R^{B}, or N(R^{A})C(O)C(O)N(R^{A})R^{B},
(3) O—C₁₋₆ alkyl,
(4) C₁₋₆ haloalkyl,
(5) O—C₁₋₆ haloalkyl,
(6) OH,
(7) halogen,
(8) CN,
(9) NO₂,
(10) N(R^{A})R^{B},
(11) C(O)N(R^{A})R^{B},
(12) C(O)R^{A},
(13) C(O)—C₁₋₆ haloalkyl,
(14) C(O)OR^{A},
(15) OC(O)N(R^{A})R^{B},
(16) SR^{A},
(17) S(O)R^{A},
(18) SO₂R^{A},
(19) SO₂N(R^{A})R^{B},
(20) N(R^{A})SO₂R^{B},
(21) N(R^{A})SO₂N(R^{A})R^{B},
(22) N(R^{A})C(O)R^{B},
(23) N(R^{A})C(O)N(R^{A})R^{B},
(24) N(R^{A})C(O)C(O)N(R^{A})R^{B}, or
(25) N(R^{A})CO₂R^{B}, and

(ii) from zero to 2 substituents are each independently:
(1) CycD,
(2) AryD,
(3) HetD,
(4) HetZ,
(5) C₁₋₆ alkyl substituted with CycD, AryD, HetD, or HetZ, or
(6) C(O)-HetZ or C(O)C(O)-HetZ;
each CycD is independently a C₃₋₈ cycloalkyl which is optionally substituted with from 1 to 4 substituents each of which is independently halogen, C₁₋₆ alkyl, OH, O—C₁₋₆ alkyl, or C₁₋₆ haloalkyl;
each AryD is independently phenyl or naphthyl, wherein the phenyl or naphthyl is optionally substituted with from 1 to 5 substituents each of which is independently any one of the substituents (1) to (25) as set forth above in part (i) of the definition of AryA;
each HetD is independently a 5- or 6-membered heteroaromatic ring containing from 1 to 4 heteroatoms independently selected from N, O and S, wherein the heteroaromatic ring is optionally substituted with from 1 to 4 substituents each of which is independently any one of the substituents (1) to (25) as set forth above in part (i) of the definition of HetA;
each HetZ is independently a 4- to 7-membered, saturated or mono-unsaturated heterocyclic ring containing at least one carbon atom and from 1 to 4 heteroatoms independently selected from N, O and S, where each S is optionally oxidized to S(O) or S(O)₂, wherein the saturated or mono-unsaturated heterocyclic ring is optionally substituted with from 1 to 4 substituents each of which is independently halogen, C₁₋₆ alkyl, C₁₋₆ haloalkyl, O—C₁₋₆ alkyl, O—C₁₋₆ haloalkyl, oxo, C(O)N(R^{A})R^{B}, C(O)C(O)N(R^{A})R^{B}, C(O)R^{A}, CO₂R^{A}, SR^{A}, S(O)R^{A}, SO₂R^{A}, or SO₂N(R^{A})R^{B};
each R^{A} is independently H or C₁₋₆ alkyl;
each R^{B} is independently H or C₁₋₆ alkyl;
each aryl is independently (i) phenyl, (ii) a 9- or 10-membered bicyclic, fused carbocyclic ring system in which at least one ring is aromatic, or (iii) an 11- to 14-membered tricyclic, fused carbocyclic ring system in which at least one ring is aromatic; and
each heteroaryl is independently (i) a 5- or 6-membered heteroaromatic ring containing from 1 to 4 heteroatoms independently selected from N, O and S, wherein each N is optionally in the form of an oxide, (ii) a 9- or 10-membered bicyclic, fused ring system, or (iii) an 11- to 14-membered tricyclic, fused ring system, wherein the fused ring system in (ii) or (iii) contains from 1 to 4 heteroatoms independently selected from N, O and S, and wherein in the fused ring system of (ii) or (iii) any one or more of the rings contain one or more of the heteroatoms, at least one ring is aromatic, each N in a ring is optionally in the form of an oxide, and each S is optionally S(O) or S(O)₂.

The present invention also includes pharmaceutical compositions containing a compound of Formula I or a pharmaceutically acceptable salt thereof. The present invention further includes methods involving compounds of Formula I for the treatment of AIDS, the delay in the onset or progression of AIDS, the prophylaxis of AIDS, the prophylaxis of infection by HIV, and the treatment of infection by HIV.

Other embodiments, aspects and features of the present invention are either further described in or will be apparent from the ensuing description, examples and appended claims.

### DETAILED DESCRIPTION OF THE INVENTION

The present invention includes compounds of Formula I above (including hydrates and solvates thereof), and pharmaceutically acceptable salts thereof. These compounds are effective inhibitors of wild-type HIV integrase (e.g., HIV-1) and may be effective inhibitors of mutant strains of HIV integrase.

A first embodiment of the present invention (alternatively referred to herein as “Embodiment E1”) is a compound of Formula I (alternatively and more simply referred to as “Compound I”), or a pharmaceutically acceptable salt thereof, wherein X is N(R³); and all other variables are as originally defined (i.e., as defined in the Summary of the Invention).

A second embodiment of the present invention (Embodiment E2) is a compound of Formula I, or a pharmaceutically acceptable salt thereof, wherein X is O; and all other variables are as originally defined.

A third embodiment of the present invention (Embodiment E3) is a compound of Formula I, or a pharmaceutically acceptable salt thereof, wherein Y is CH₂, CH(CH₃), CH₂CH₂, CH(CH₃)CH(CH₃), CH₂CH(CH₃), or CH(CH₃)CH₂; and all other variables are as originally defined or as defined in either of Embodiments E1 or E2.

A fourth embodiment of the present invention (Embodiment E4) is a compound of Formula I, or a pharmaceutically acceptable salt thereof, wherein Y is CH₂CH₂; and all other variables are as originally defined or as defined in either of Embodiments E1 or E2.

A fifth embodiment of the present invention (Embodiment E5) is a compound of Formula I, or a pharmaceutically acceptable salt thereof, wherein R¹ is:

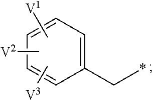

the asterisk * denotes the point of attachment of R¹ to the rest of the compound;
V¹ and V² are each independently: (1) H, (2) C1-4 alkyl, (3) OH, (4) O—C1-4 alkyl, (5) C1-4haloalkyl, (6) O—C1-4 haloalkyl, (7) halogen, (8) CN, (9) N(R^{A})R^{B}, (10) C(O)N(R^{A})R^{B}, (11) C(O)R^{A}, (12) C(O)OR^{A}, (13) SR^{A}, (14) S(O)R^{A}, (15) SO₂R^{A}, (16) N(R^{A})SO2R^{B}, (17) N(R^{A})SO2N(R^{A})R^{B}, (18) N(R^{A})C(O)R^{B}, (19) N(R^{A})C(O)C(O)N(R^{A})R^{B}, (20) HetD, (21) HetZ, or (22) C(O)-HetZ;
or alternatively V¹ and V² are respectively located on adjacent carbons in the phenyl ring and together form methylenedioxy or ethylenedioxy;
V³ is: (1) H, (2) C₁₋₄ alkyl, (3) O—C₁₋₄ alkyl, (4) C₁₋₄ haloalkyl, (5) O—C₁₋₄ haloalkyl, or (6) halogen;
   and all other variables are as originally defined or as defined in any one of the foregoing embodiments.

In a first aspect of Embodiment E5, HetD and HetZ in V¹ and V² are defined as follows:

HetD is a 5- or 6-membered heteroaromatic ring containing a total of from 1 to 3 heteroatoms independently selected from 1 to 3 N atoms, from zero to 1 O atom, and from zero to 1 S atom, wherein the heteroaromatic ring is optionally substituted with from 1 to 3 substituents each of which is independently C₁₋₄ alkyl, OH, O—C₁₋₄ alkyl, halogen, CN, C(O)N(R^{A})R^{B}, C(O)R^{A}, C(O)OR^{A}, or SO₂R^{A};

HetZ is a 5- or 6-membered saturated heterocyclic ring containing a total of from 1 to 2 heteroatoms selected from 1 to 2 N atoms, zero to 1 O atom, and zero to 1 S atom, wherein the S atom is optionally S(O) or SO₂, wherein the saturated heterocyclic ring is optionally substituted with from 1 to 2 substituents each of which is independently C₁₋₄ alkyl, oxo, C(O)N(R^{A})R^{B}, C(O)R^{A}, CO₂R^{A}, or SO₂R^{A};

and with the proviso that when HetZ is attached to the rest of the compound via the C(O) moiety, then HetZ is attached to the C(O) via a ring N atom;

and all other variables are as originally defined in Embodiment E5.

In a second aspect of Embodiment E5, each R^{A} is independently H or C₁₋₄ alkyl; each R^{B} is independently H or C₁₋₄ alkyl; and all other variables are as originally defined in Embodiment E5.

In a third aspect of Embodiment E5, HetD and HetZ are as defined in the first aspect; R^{A} and R^{B} are as defined in the second aspect; and all other variables are as originally defined in Embodiment E5.

A sixth embodiment of the present invention (Embodiment E6) is a compound of Formula I, or a pharmaceutically acceptable salt thereof, wherein R¹ is:

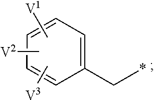

the asterisk * denotes the point of attachment of R¹ to the rest of the compound;
V¹ and V² are each independently: (1) H, (2) CH₃, (3) CF₃, (4) OH, (5) OCH₃, (6) Cl, Br, or F, (7) CN, (8) C(O)NH₂, (9) C(O)NH(CH₃), (10) C(O)N(CH₃)₂, or (11) SO₂CH₃;
V³ is H, Cl, Br, F, CH₃, or OCH₃;
   and all other variables are as originally defined or as defined in any one of the foregoing embodiments.

A seventh embodiment of the present invention (Embodiment E7) is a compound of Formula I, or a pharmaceutically acceptable salt thereof, wherein R¹ is 4-fluorobenzyl, 3-chloro-4-fluorobenzyl, or 4-fluoro-3-methylbenzyl; and all other variables are as originally defined or as defined in any one of the foregoing embodiments.

An eighth embodiment of the present invention (Embodiment E8) is a compound of Formula I, or a pharmaceutically acceptable salt thereof, wherein:
R² is H, C₁₋₄ alkyl, or CH(R^{K})R^{L};
R^{K} is H or C₁₋₄ alkyl;
R^{L} is C(R^{T})═CH₂,

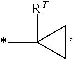

1-hydroxyethyl, 2-hydroxyethyl, 1,2-dihydroxyethyl, 2-amino-1-hydroxyethyl, or 1-amino-2-hydroxyethyl;
R^{T} is H or C₁₋₄ alkyl;
R³ is H, C₁₋₄ alkyl, or CH(R^{Q})—C₁₋₄ alkenyl;
R^{Q} is H or C₁₋₄ alkyl;
each R⁴ is independently H or C₁₋₄ alkyl;
alternatively, when X is N(R³) and R² and R³ together with the atoms to which they are attached form an azacycloalkyl ring of Formula Ia, then each R^{M} is independently H, C₁₋₄ alkyl, OH, C₁₋₄ alkoxy, or oxo which is formed together with the R^{M′} attached to the same carbon; with the proviso that at least 2 of the R^{M} groups are H; and for each R^{M} which is other than oxo, R^{M′} is H or C₁₋₄ alkyl, with the proviso that when R^{M} is H, then R^{M′} attached to the same carbon is also H;
   and all other variables are as originally defined or as defined in any one of the foregoing embodiments.

A ninth embodiment of the present invention (Embodiment E9) is a compound of Formula I, or a pharmaceutically acceptable salt thereof, wherein:
R² is H, CH₃, CH₂—CH═CH₂, or CH(CH₃)—CH═CH₂;
R³ is H, CH₃, (CH₂)₁₋₃—CH═CH₂, or CH(CH₃)(CH₂)₁₋₂—CH═CH₂;
each R⁴ is independently H or CH₃;
alternatively, when X is N(R³) and R² and R³ together with the atoms to which they are attached form an azacycloalkyl ring of Formula Ia, then each R^{M} is independently H, CH₃, OH, OCH₃, or oxo which is formed together with the R^{M′} attached to the same carbon; with the proviso that at least 2 of the R^{M} groups are H; and for each R^{M} which is other than oxo, R^{M′} is H.
   and all other variables are as originally defined or as defined in any one of the foregoing embodiments.

A first class of compounds of the present invention (alternatively referred to herein as Class C1) includes compounds of Formula II and pharmaceutically acceptable salts thereof:

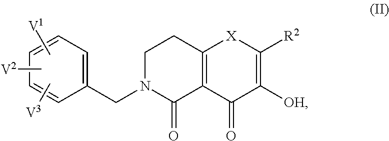

wherein:
X is O or N(R³);
R² is H, C₁₋₄ alkyl, or CH(R^{K})R^{L};
R^{K} is H or C₁₋₄ alkyl;
R^{L} is C(R^{T})═CH₂ or

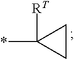

R^{T} is H or C₁₋₄ alkyl;
R³ is H, C₁₋₄ alkyl, or CH(R^{Q})—C₁₋₄ alkenyl;
R^{Q} is H or C₁₋₄ alkyl;
alternatively, when X is N(R³), R² and R³ together with the atoms to which they are attached form an azacycloalkyl ring such that the compound of Formula II is a compound of Formula IIa:

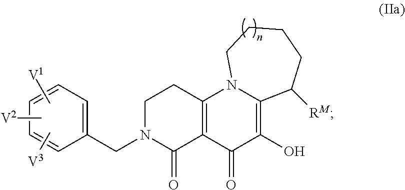

R^{M} is independently H or C₁₋₄ alkyl;
n is zero or 1;
V¹ and V² are each independently:
   (1) H,
   (2) C₁₋₄ alkyl,
   (3) OH,
   (4) O—C₁₋₄ alkyl,
   (5) C₁₋₄ haloalkyl,
   (6) O—C₁₋₄ haloalkyl,
   (7) halogen,
   (8) CN,
   (9) N(R^{A})R^{B},
   (10) C(O)N(R^{A})R^{B},
   (11) C(O)R^{A},
   (12) C(O)OR^{A},
   (13) SR^{A},
   (14) S(O)R^{A},
   (15) SO₂R^{A},
   (16) N(R^{A})SO₂R^{B},
   (17) N(R^{A})SO₂N(R^{A})R^{B},
   (18) N(R^{A})C(O)R^{B},
   (19) N(R^{A})C(O)C(O)N(R^{A})R^{B},
   (20) HetD,
   (21) HetZ, or
   (22) C(O)-HetZ, wherein:
      HetD is a 5- or 6-membered heteroaromatic ring containing a total of from 1 to 3 heteroatoms independently selected from 1 to 3 N atoms, from zero to 1 O atom, and from zero to 1 S atom, wherein the heteroaromatic ring is optionally substituted with from 1 to 3 substituents each of which is independently C₁₋₄ alkyl, OH, O—C₁₋₄ alkyl, halogen, CN, C(O)N(R^{A})R^{B}, C(O)R^{A}, C(O)OR^{A}, or SO₂R^{A},
      HetZ is a 5- or 6-membered saturated heterocyclic ring containing a total of from 1 to 2 heteroatoms selected from 1 to 2 N atoms, zero to 1 O atom, and zero to 1 S atom, wherein the S atom is optionally S(O) or SO₂, wherein the saturated heterocyclic ring is optionally substituted with from 1 to 2 substituents each of which is independently C₁₋₄ alkyl, oxo, C(O)N(R^{A})R^{B}, C(O)R^{A}, CO₂R^{A}, or SO₂R^{A},
      and with the proviso that when HetZ is attached to the rest of the compound via the C(O) moiety, then HetZ is attached to the C(O) via a ring N atom;
or alternatively V¹ and V² are respectively located on adjacent carbons in the phenyl ring and together form methylenedioxy or ethylenedioxy;
V³ is:
   (1) H,
   (2) C₁₋₄ alkyl,
   (3) O—C₁₋₄ alkyl,
   (4) C₁₋₄ haloalkyl,
   (5) O—C₁₋₄ haloalkyl, or
   (6) halogen;
each R^{A} is independently H or C₁₋₄ alkyl; and
each R^{B} is independently H or C₁₋₄ alkyl.

A first sub-class of the first class (alternatively referred to herein as “Sub-class C1-S1”) includes compounds of Formula II and pharmaceutically acceptable salts thereof, wherein X is N(R³); and all of the other variables are as originally defined in Class C1.

A second sub-class of the first class (Sub-class C1-S2) includes compounds of Formula II and pharmaceutically acceptable salts thereof, wherein X is O; and all of the other variables are as originally defined in Class C1.

A third sub-class of the first class (Sub-class C1-S3) includes compounds of Formula II and pharmaceutically acceptable salts thereof, wherein:

### R² is H, CH₃, CH₂—CH═CH₂, or CH(CH₃)—CH═CH₂;

### R³ is H, CH₃, (CH₂)₁₋₃—CH═CH₂, or CH(CH₃)(CH₂)₁₋₂—CH═CH₂;

### R^{M} is H or CH₃.

V¹ and V² are each independently:

(1) H,

(2) CH₃,

(3) CF₃,

(4) OH,

(5) OCH₃,

(6) Cl, Br, or F,

(7) CN,

(8) C(O)NH₂,

(9) C(O)NH(CH₃),

(10) C(O)N(CH₃)₂, or

(11) SO₂CH₃; and

### V³ is H, Cl, Br, F, CH₃, or OCH₃;

and all of the other variables are as originally defined in Class C1.

A second class of compounds of the present invention (Class C2) is a compound selected from the group consisting of

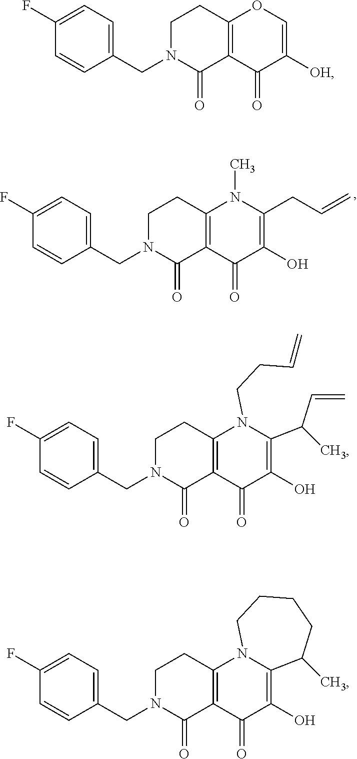

and pharmaceutically acceptable salts thereof.

Another embodiment of the present invention is a compound of Formula I, or a pharmaceutically acceptable salt thereof, as originally defined or as defined in any of the foregoing embodiments, sub-embodiments, aspects, classes, or sub-classes, wherein the compound or its salt is in a substantially pure form. As used herein “substantially pure” means suitably at least about 60 wt. %, typically at least about 70 wt. %, preferably at least about 80 wt. %, more preferably at least about 90 wt. % (e.g., from about 90 wt. % to about 99 wt. %), even more preferably at least about 95 wt % (e.g., from about 95 wt. % to about 99 wt. %, or from about 98 wt. % to 100 wt. %), and most preferably at least about 99 wt. % (e.g., 100 wt. %) of a product containing a compound of Formula I or its salt (e.g., the product isolated from a reaction mixture affording the compound or salt) consists of the compound or salt. The level of purity of the compounds and salts can be determined using a standard method of analysis such as thin layer chromatography, gel electrophoresis, high performance liquid chromatography, and/or mass spectrometry. If more than one method of analysis is employed and the methods provide experimentally significant differences in the level of purity determined, then the method providing the highest purity level governs. A compound or salt of 100% purity is one which is free of detectable impurities as determined by a standard method of analysis. With respect to a compound of the invention which has one or more asymmetric centers and can occur as mixtures of stereoisomers, a substantially pure compound can be either a substantially pure mixture of the stereoisomers or a substantially pure individual diastereomer or enantiomer.

The present invention also includes prodrugs of the compounds of Formula I. The term “prodrug” refers to a derivative of a compound of Formula I, or a pharmaceutically acceptable salt thereof, which is converted in vivo into Compound I. Prodrugs of compounds of Formula I can exhibit enhanced solubility, absorption, and/or lipophilicity compared to the compounds per se, thereby resulting in increased bioavailability and efficacy. The in vivo conversion of the prodrug can be the result of an enzyme-catalyzed chemical reaction, a metabolic chemical reaction, and/or a spontaneous chemical reaction (e.g., solvolysis). When the compound contains, for example, a hydroxy group, the prodrug can be a derivative of the hydroxy group such as an ester (—OC(O)R), a carbonate ester (—OC(O)OR), a phosphate ester (—O—P(═O)(OH)₂), or an ether (—OR). Other examples include the following: When the compound of Formula I contains a carboxylic acid group, the prodrug can be an ester or an amide, and when the compound of Formula I contains a primary amino group or another suitable nitrogen that can be derivatized, the prodrug can be an amide, carbamate, urea, imine, or a Mannich base. One or more functional groups in Compound I can be derivatized to provide a prodrug thereof. Conventional procedures for the selection and preparation of suitable prodrug derivatives are described, for example, in Design of Prodrugs, edited by H. Bundgaard, Elsevier, 1985; J. J. Hale et al., J. Med. Chem. 2000, vol. 43, pp. 1234-1241; C. S. Larsen and J. Ostergaard, “Design and application of prodrugs” in: Textbook of Drug Design and Discovery, 3^{rd} edition, edited by C, S. Larsen, 2002, pp. 410-458; and Beaumont et al., Current Drug Metabolism 2003, vol. 4, pp. 461-458; the disclosures of each of which are incorporated herein by reference in their entireties.

Other embodiments of the present invention include the following:

(a) A pharmaceutical composition comprising an effective amount of a compound of Formula I as defined above, or a prodrug or pharmaceutically acceptable salt thereof, and a pharmaceutically acceptable carrier.

(b) A pharmaceutical composition which comprises the product prepared by combining (e.g., mixing) an effective amount of a compound of Formula I as defined above, or a prodrug or pharmaceutically acceptable salt thereof, and a pharmaceutically acceptable carrier.

(c) The pharmaceutical composition of (a) or (b), further comprising an effective amount of an anti-HIV agent selected from the group consisting of HIV antiviral agents, immunomodulators, and anti-infective agents.

(d) The pharmaceutical composition of (c), wherein the anti-HIV agent is an antiviral selected from the group consisting of HIV protease inhibitors, HIV reverse transcriptase inhibitors (nucleoside or non-nucleoside), HIV integrase inhibitors, HIV fusion inhibitors, and HIV entry inhibitors.

(e) A combination which is (i) a compound of Formula I as defined above, or a prodrug or pharmaceutically acceptable salt thereof, and (ii) an anti-HIV agent selected from the group consisting of HIV antiviral agents, immunomodulators, and anti-infective agents; wherein Compound I and the anti-HIV agent are each employed in an amount that renders the combination effective for inhibition of HIV integrase, for treatment or prophylaxis of infection by HIV, or for treatment, prophylaxis of, or delay in the onset or progression of AIDS.

(f) The combination of (e), wherein the anti-HIV agent is an antiviral selected from the group consisting of HIV protease inhibitors, HIV reverse transcriptase inhibitors (nucleoside or non-nucleoside), HIV integrase inhibitors, HIV fusion inhibitors, and HIV entry inhibitors.

(g) A method for the inhibition of HIV integrase in a subject in need thereof which comprises administering to the subject an effective amount of a compound of Formula I or a prodrug or pharmaceutically acceptable salt thereof.

(h) A method for the prophylaxis or treatment of infection by HIV (e.g., HIV-1) in a subject in need thereof which comprises administering to the subject an effective amount of a compound of Formula I or a prodrug or pharmaceutically acceptable salt thereof.

(i) The method of (h), wherein the compound of Formula I is administered in combination with an effective amount of at least one other HIV antiviral selected from the group consisting of HIV protease inhibitors, HIV integrase inhibitors, non-nucleoside HIV reverse transcriptase inhibitors, nucleoside HIV reverse transcriptase inhibitors, HIV fusion inhibitors, and HIV entry inhibitors.

(j) A method for the prophylaxis, treatment or delay in the onset or progression of AIDS in a subject in need thereof which comprises administering to the subject an effective amount of a compound of Formula I or a prodrug or pharmaceutically acceptable salt thereof.

(k) The method of (j), wherein the compound is administered in combination with an effective amount of at least one other HIV antiviral selected from the group consisting of HIV protease inhibitors, HIV integrase inhibitors, non-nucleoside HIV reverse transcriptase inhibitors, nucleoside HIV reverse transcriptase inhibitors, HIV fusion inhibitors, and HIV entry inhibitors.

(l) A method for the inhibition of HIV integrase in a subject in need thereof which comprises administering to the subject the pharmaceutical composition of (a), (b), (c) or (d) or the combination of (e) or (f).

(m) A method for the prophylaxis or treatment of infection by HIV (e.g., HIV-1) in a subject in need thereof which comprises administering to the subject the pharmaceutical composition of (a), (b), (c) or (d) or the combination of (e) or (f).

(n) A method for the prophylaxis, treatment, or delay in the onset or progression of AIDS in a subject in need thereof which comprises administering to the subject the pharmaceutical composition of (a), (b), (c) or (d) or the combination of (e) or (f).

(o) A method for the inhibition of XMRV in a subject in need thereof which comprises administering to the subject an effective amount of a compound of Formula I or a prodrug or pharmaceutically acceptable salt thereof, wherein the compound or its prodrug or salt is optionally administered as a component in a composition further comprising a pharmaceutically acceptable carrier.

(p) A method for the prophylaxis or treatment of infection by XMRV in a subject in need thereof which comprises administering to the subject an effective amount of a compound of Formula I or a prodrug or pharmaceutically acceptable salt thereof, wherein the compound or its prodrug or salt is optionally administered as a component in a composition further comprising a pharmaceutically acceptable carrier.

(q) A method for the prophylaxis, treatment or delay in the onset or progression of a disease or condition caused by XMRV infection (e.g., prostate cancer or chronic fatigue syndrome) in a subject in need thereof which comprises administering to the subject an effective amount of a compound of Formula I or a prodrug or pharmaceutically acceptable salt thereof, wherein the compound or its prodrug or salt is optionally administered as a component in a composition further comprising a pharmaceutically acceptable carrier.

(r) A method for the inhibition of HTLV in a subject in need thereof which comprises administering to the subject an effective amount of a compound of Formula I or a prodrug or pharmaceutically acceptable salt thereof, wherein the compound or its prodrug or salt is optionally administered as a component in a composition further comprising a pharmaceutically acceptable carrier.

(s) A method for the prophylaxis or treatment of infection by HTLV in a subject in need thereof which comprises administering to the subject an effective amount of a compound of Formula I or a prodrug or pharmaceutically acceptable salt thereof, wherein the compound or its prodrug or salt is optionally administered as a component in a composition further comprising a pharmaceutically acceptable carrier.

(t) A method for the prophylaxis, treatment or delay in the onset or progression of a disease or condition caused by HTLV infection (e.g., T-cell leukemia or T-cell lymphoma) in a subject in need thereof which comprises administering to the subject an effective amount of a compound of Formula I or a prodrug or pharmaceutically acceptable salt thereof, wherein the compound or its prodrug or salt is optionally administered as a component in a composition further comprising a pharmaceutically acceptable carrier.

The present invention also includes a compound of Formula I, or a prodrug or pharmaceutically acceptable salt thereof, (i) for use in, (ii) for use as a medicament for, or (iii) for use in the preparation of a medicament for: (a) therapy (e.g., of the human body), (b) medicine, (c) inhibition of HIV integrase, (d) treatment or prophylaxis of infection by HIV, (e) treatment, prophylaxis of, or delay in the onset or progression of AIDS, (f) inhibition of XMRV, (g) treatment or prophylaxis of infection by XMRV, (h) treatment or prophylaxis or delay in the onset or progression of a disease or condition caused by XMRV infection, (i) inhibition of HTLV, (j) treatment or prophylaxis of infection by HTLV, (k) treatment or prophylaxis or delay in the onset or progression of a disease or condition caused by HTLV infection. In these uses, the compounds of the present invention can optionally be employed in combination with one or more anti-HIV agents selected from HIV antiviral agents, anti-infective agents, and immunomodulators.

Additional embodiments of the invention include the pharmaceutical compositions, combinations and methods set forth in (a)-(t) above and the uses (i)(a)-(k) through (iii)(a)-(k) set forth in the preceding paragraph, wherein the compound of the present invention employed therein is a compound of one of the embodiments, sub-embodiments, aspects, features, classes, or sub-classes described above. In all of these embodiments etc., the compound may optionally be used in the form of a prodrug or pharmaceutically acceptable salt.

Additional embodiments of the present invention include each of the pharmaceutical compositions, combinations, methods and uses set forth in the preceding paragraphs, wherein the compound of the present invention or a salt or prodrug thereof employed therein is substantially pure. With respect to a pharmaceutical composition comprising a compound of Formula I or its prodrug or salt and a pharmaceutically acceptable carrier and optionally one or more excipients, it is understood that the term “substantially pure” is in reference to a compound of Formula I or its prodrug or salt per se.

Still additional embodiments of the present invention include the pharmaceutical compositions, combinations and methods set forth in (a)-(n) above and the uses (i)(a)-(e) through (iii)(a)-(e) set forth above, wherein the HIV of interest is HIV-1. Thus, for example, in the pharmaceutical composition (d), the compound of Formula I is employed in an amount effective against HIV-1 and the anti-HIV agent is an HIV-1 antiviral selected from the group consisting of HIV-1 protease inhibitors, HIV-1 reverse transcriptase inhibitors, HIV-1 integrase inhibitors, HIV-1 fusion inhibitors and HIV-1 entry inhibitors.

As used herein, the term “alkyl” refers to a monovalent straight or branched chain, saturated aliphatic hydrocarbon radical having a number of carbon atoms in the specified range. Thus, for example, “C₁₋₆ alkyl” (or “C₁-C₆ alkyl”) refers to any of the hexyl alkyl and pentyl alkyl isomers as well as n-, iso-, sec- and t-butyl, n- and iso-propyl, ethyl and methyl. As another example, “C₁₋₄ alkyl” refers to n-, iso-, sec- and t-butyl, n- and isopropyl, ethyl and methyl.

The term “halogen” (or “halo”) refers to fluorine, chlorine, bromine and iodine (alternatively referred to as fluoro, chloro, bromo, and iodo).

The term “haloalkyl” refers to an alkyl group as defined above in which one or more of the hydrogen atoms have been replaced with a halogen (i.e., F, Cl, Br and/or I). Thus, for example, “C₁₋₆ haloalkyl” (or “C₁-C₆ haloalkyl”) refers to a C₁ to C₆ linear or branched alkyl group as defined above with one or more halogen substituents. The term “fluoroalkyl” has an analogous meaning except that the halogen substituents are restricted to fluoro. Suitable fluoroalkyls include the series (CH₂)₀₋₄CF₃ (i.e., trifluoromethyl, 2,2,2-trifluoroethyl, 3,3,3-trifluoro-n-propyl, etc.). A fluoroalkyl of particular interest is CF₃.

The term “C(O)” refers to carbonyl. The terms “S(O)₂” and “SO₂” each refer to sulfonyl. The term “S(O)” refers to sulfinyl.

An asterisk (“*”) as the end of an open bond in a chemical group denotes the point of attachment of the group to the rest of the compound.

The term “aryl” refers to (i) phenyl, (ii) a 9- or I O-membered bicyclic, fused carbocyclic ring system in which at least one ring is aromatic, or (iii) an 11- to 14-membered tricyclic, fused carbocyclic ring system in which at least one ring is aromatic. Suitable aryls include, for example, phenyl, naphthyl, 1,2,3,4-tetrahydronaphthyl, indenyl, indanyl, acenaphthenyl and fluorenyl. A class of aryls suitable for use in the present invention is phenyl, naphthyl, and indenyl. Another class of suitable aryls is phenyl and naphthyl (e.g., see AryD). A particularly suitable aryl is phenyl.

The term “heteroaryl” refers to (i) a 5- or 6-membered heteroaromatic ring containing from 1 to 4 heteroatoms independently selected from N, O and S, wherein each N is optionally in the form of an oxide, (ii) a 9- or 10-membered bicyclic, fused ring system, or (iii) an 11- to 14-membered tricyclic, fused ring system, wherein the fused ring system in (ii) or (iii) contains from 1 to 4 heteroatoms independently selected from N, O and S, and wherein in the fused ring system of (ii) or (iii) any one or more of the rings contain one or more of the heteroatoms, at least one ring is aromatic, each N in a ring is optionally in the form of an oxide, and each S is optionally S(O) or S(O)₂. Suitable heteroaryls include, for example, pyridinyl, pyrrolyl, pyrazinyl, pyrimidinyl, pyridazinyl, triazinyl, thienyl, furanyl, imidazolyl, pyrazolyl, triazolyl, tetrazolyl, oxazolyl, isooxazolyl, oxadiazolyl, quinolinyl, tetrahydroquinolinyl, isoquinolinyl, tetrahydroisoquinolinyl, indolyl, isoindolyl, benzodioxolyl, benzopiperidinyl, benzisoxazolyl, benzoxazolyl, chromenyl, chromanyl, isochromanyl, cinnolinyl, quinazolinyl, benzothienyl, benzofuranyl, imidazo[1,2-a]pyridinyl, benzotriazolyl, dihydroindolyl, dihydroisoindolyl, indazolyl, indolinyl, isoindolinyl, quinoxalinyl, quinazolinyl, 2,3-dihydrobenzofuranyl, 2,3-dihydrobenzo-1,4-dioxinyl (i.e.,

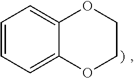

, benzo-1,3-dioxolyl (i.e.,

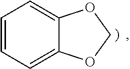

thiazolyl, and isothiazolyl.

A class of heteroaryls suitable for use in the present invention (e.g., see HetD) consists of 5- and 6-membered heteroaromatic rings containing from 1 to 4 heteroatoms independently selected from N, O and S, wherein each N is optionally in the form of an oxide. Heteroaryls belonging to this class include pyridinyl, pyrrolyl, pyrazinyl, pyrimidinyl, pyridazinyl, triazinyl, furanyl, imidazolyl, pyrazolyl, triazolyl, tetrazolyl, oxazolyl, isooxazolyl, and oxadiazolyl. A sub-class of heteroaryls suitable for use in the present invention consists of

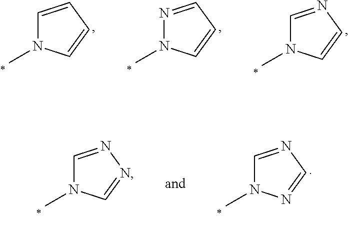

Another suitable class consists of 5- and 6-membered heteroaromatic rings containing a total of from 1 to 3 heteroatoms independently selected from 1 to 3 N atoms, from zero to 1 O atom, and from zero to 1 S atom.

The term “saturated or mono-unsaturated heterocyclic ring” refers to (i) a 4- to 7-membered, saturated or mono-unsaturated heterocyclic ring containing at least one carbon atom and from 1 to 4 heteroatoms independently selected from N, O and S, where each S is optionally oxidized to S(O) or S(O)₂ or (ii) a 6- to 10-membered saturated or mono-unsaturated, bridged or fused heterobicyclic ring containing from 1 to 4 heteroatoms independently selected from N, O and S, where each S is optionally oxidized to S(O) or S(O)₂. Suitable saturated heterocycles include, for example, azetidinyl, pyrrolidinyl, imidazolinyl, tetrahydrofuranyl, tetrahydrothienyl, pyrazolidinyl, piperidinyl, piperazinyl, hexahydropyrimidinyl, morpholinyl, thiomorpholinyl, thiazolidinyl, isothiazolidinyl, oxazolidinyl, isoxazolidinyl, thiazinanyl, azepanyl, diazepanyl, thiazepanyl and thiadiazepanyl. A class of suitable saturated or mono-unsaturated heterocyclic rings are the 4- to 7-membered rings containing at least one carbon atom and from 1 to 4 heteroatoms independently selected from N, O and S, where each S is optionally oxidized to S(O) or S(O)₂ (e.g., see HetZ). Another suitable class consists of 5- or 6-membered saturated heterocyclic rings containing a total of from 1 to 2 heteroatoms selected from 1 to 2 N atoms, zero to 1 O atom, and zero to 1 S atom, wherein the S atom is optionally S(O) or SO₂. Suitable mono-unsaturated heterocyclic rings include those corresponding to the saturated heterocyclic rings listed in the preceding sentence in which a single bond is replaced with a double bond (e.g., a carbon-carbon single bond is replaced with a carbon-carbon double bond).

It is understood that the specific rings and ring systems suitable for use in the present invention are not limited to those listed in the preceding paragraphs. These rings and ring systems are merely representative.

Unless expressly stated to the contrary in a particular context, any of the various cyclic rings and ring systems described herein may be attached to the rest of the compound at any ring atom (i.e., any carbon atom or any heteroatom) provided that a stable compound results.

Unless expressly stated to the contrary, all ranges cited herein are inclusive. For example, a heteroaromatic ring described as containing from “1 to 4 heteroatoms” means the ring can contain 1, 2, 3 or 4 heteroatoms. It is also to be understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, 3 heteroatoms, and 4 heteroatoms. As another example, a phenyl or naphthyl (see, e.g., the definition of AryA) described as optionally substituted with “from 1 to 5 substituents” is intended to include as aspects thereof, a phenyl or naphthyl substituted with 1 to 5 substituents, 2 to 5 substituents, 3 to 5 substituents, 4 to 5 substituents, 5 substituents, 1 to 4 substituents, 2 to 4 substituents, 3 to 4 substituents, 4 substituents, 1 to 3 substituents, 2 to 3 substituents, 3 substituents, 1 to 2 substituents, 2 substituents, and 1 substituent.

When any variable (e.g., R^{A} or R^{B}) occurs more than one time in any constituent or in Formula I or in any other formula depicting and describing compounds of the present invention, its definition on each occurrence is independent of its definition at every other occurrence. Also, combinations of substituents and/or variables are permissible only if such combinations result in stable compounds.

Unless expressly stated to the contrary, substitution by a named substituent is permitted on any atom in a ring provided such ring substitution is chemically allowed and results in a stable compound.

In the definition of R^{M}, an R^{M} can form methylene together with an R^{M} on an adjacent ring carbon to provide fused cyclopropyl. This feature of the definition is illustrated as follows:

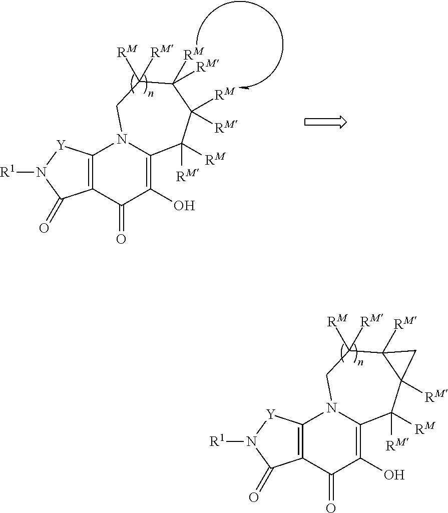

As would be recognized by one of ordinary skill in the art, certain of the compounds of the present invention can exist as tautomers. All tautomeric forms of these compounds, whether isolated individually or in mixtures, are within the scope of the present invention. For example, in instances where a hydroxy (—OH) substituent is permitted on a heteroaromatic ring and keto-enol tautomerism is possible, it is understood that the substituent might in fact be present, in whole or in part, in the keto form, as exemplified here for a hydroxypyridinyl substituent:

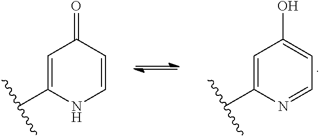

Compounds of the present invention having a hydroxy substituent on a carbon atom of a heteroaromatic ring are understood to include compounds in which only the hydroxy is present, compounds in which only the tautomeric keto form (i.e., an oxo substitutent) is present, and compounds in which the keto and enol forms are both present.

A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject). The compounds of the present invention are limited to stable compounds embraced by Formula I.

As a result of the selection of substituents and substituent patterns, certain compounds of the present invention can have asymmetric centers and can occur as mixtures of stereoisomers, or as individual diastereomers, or enantiomers. All isomeric forms of these compounds, whether individually or in mixtures, are within the scope of the present invention.

All solvates and hydrates of compounds of Formula I are within the scope of the present invention.

The atoms in a compound of Formula I may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I can be prepared without undue experimentation by conventional techniques well known to those skilled in the art or by processes analogous to those described in the Schemes and Examples herein using appropriate isotopically-enriched reagents and/or intermediates.

The methods of the present invention involve the use of compounds of Formula in the inhibition of HIV integrase (e.g., wild type HIV-1 and/or mutant strains thereof), the prophylaxis or treatment of infection by human immunodeficiency virus (HIV) and the prophylaxis, treatment or delay in the onset or progression of consequent pathological conditions such as AIDS. Prophylaxis of AIDS, treating AIDS, delaying the onset or progression of AIDS, or treating or prophylaxis of infection by HIV is defined as including, but not limited to, treatment of a wide range of states of HIV infection: AIDS, ARC (AIDS related complex), both symptomatic and asymptomatic, and actual or potential exposure to HIV. For example, the present invention can be employed to treat infection by HIV after suspected past exposure to HIV by such means as blood transfusion, exchange of body fluids, bites, accidental needle stick, or exposure to patient blood during surgery. As another example, the present invention can also be employed to prevent transmission of HIV from a pregnant female infected with HIV to her unborn child or from an HIV-infected female who is nursing (i.e., breast feeding) a child to the child via administration of an effective amount of Compound I or a prodrug or pharmaceutically acceptable salt thereof.

The compounds can be administered in the form of pharmaceutically acceptable salts. The term “pharmaceutically acceptable salt” refers to a salt which possesses the effectiveness of the parent compound and which is not biologically or otherwise undesirable (e.g., is neither toxic nor otherwise deleterious to the recipient thereof). Suitable salts include acid addition salts which may, for example, be formed by mixing a solution of the compound of the present invention with a solution of a pharmaceutically acceptable acid such as hydrochloric acid, sulfuric acid, acetic acid, or benzoic acid. When compounds employed in the present invention carry an acidic moiety (e.g., —COOH or a phenolic group), suitable pharmaceutically acceptable salts thereof can include alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

The term “administration” and variants thereof (e.g., “administering” a compound) in reference to a compound of Formula I mean providing the compound or a prodrug or salt of the compound to the individual in need of treatment or prophylaxis. When a compound or a prodrug or salt thereof is provided in combination with one or more other active agents (e.g., antiviral agents useful for treating or prophylaxis of HIV infection or AIDS), “administration” and its variants are each understood to include provision of the compound or prodrug or salt thereof and other agents at the same time or at different times. When the agents of a combination are administered at the same time, they can be administered together in a single composition or they can be administered separately.

As used herein, the term “composition” is intended to encompass a product comprising the specified ingredients, as well as any product which results, directly or indirectly, from combining the specified ingredients.

By “pharmaceutically acceptable” is meant that the ingredients of the pharmaceutical composition must be compatible with each other and not deleterious to the recipient thereof.

The term “subject” as used herein refers to an animal, preferably a mammal, most preferably a human, who has been the object of treatment, observation or experiment.

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active compound sufficient to inhibit HIV integrase (wild type and/or mutant strains thereof) and thereby elicit the response being sought (i.e., an “inhibition effective amount”). When the active compound (i.e., active ingredient) is administered as the salt, references to the amount of active ingredient are to the free form (i.e., the non-salt and non-prodrug form) of the compound.

In the method of the present invention (i.e., inhibiting HIV integrase, treating or prophylaxis of HIV infection or treating, prophylaxis of, or delaying the onset or progression of AIDS), the compounds of Formula I, optionally in the form of a salt or a prodrug, can be administered by any means that produces contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered orally, parenterally (including subcutaneous injections, intravenous, intramuscular, intrasternal injection or infusion techniques), by inhalation spray, or rectally, in the form of a unit dosage of a pharmaceutical composition containing an effective amount of the compound and conventional non-toxic pharmaceutically acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions for use in the present invention and of ingredients suitable for use in said compositions is provided in Remington's Pharmaceutical Sciences, 18^{th} edition, edited by A. R. Gennaro, Mack Publishing Co., 1990 and in Remington—The Science and Practice of Pharmacy, 21st edition, Lippincott Williams & Wilkins, 2005.

The compounds of Formula I can be administered orally in a dosage range of 0.001 to 1000 mg/kg of mammal (e.g., human) body weight per day in a single dose or in divided doses. One preferred dosage range is 0.01 to 500 mg/kg body weight per day orally in a single dose or in divided doses. Another preferred dosage range is 0.1 to 100 mg/kg body weight per day orally in single or divided doses. For oral administration, the compositions can be provided in the form of tablets or capsules containing 1.0 to 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

As noted above, the present invention is also directed to use of a compound of Formula I with one or more anti-HIV agents. An “anti-HIV agent” is any agent which is directly or indirectly effective in the inhibition of HIV reverse transcriptase or another enzyme required for HIV replication or infection, the treatment or prophylaxis of HIV infection, and/or the treatment, prophylaxis or delay in the onset or progression of AIDS. It is understood that an anti-HIV agent is effective in treating, preventing, or delaying the onset or progression of HIV infection or AIDS and/or diseases or conditions arising therefrom or associated therewith. For example, the compounds of this invention may be effectively administered, whether at periods of pre-exposure and/or post-exposure, in combination with effective amounts of one or more anti-HIV agents selected from HIV antiviral agents, immunomodulators, antiinfectives, or vaccines useful for treating HIV infection or AIDS. Suitable HIV antivirals for use in combination with the compounds of the present invention include, for example, those listed in Table A as follows:

TABLE A

| Name | Type |
|---|---|
| abacavir, ABC, Ziagen ® | nRTI |
| abacavir + lamivudine, Epzicom ® | nRTI |
| abacavir + lamivudine + zidovudine, Trizivir ® | nRTI |
| amprenavir, Agenerase ® | PI |
| atazanavir, Reyataz ® | PI |
| AZT, zidovudine, azidothymidine, Retrovir ® | nRTI |
| darunavir, Prezista ® | PI |
| ddC, zalcitabine, dideoxycytidine, Hivid ® | nRTI |
| ddI, didanosine, dideoxyinosine, Videx ® | nRTI |
| ddI (enteric coated), Videx EC ® | nRTI |
| delavirdine, DLV, Rescriptor ® | nnRTI |
| efavirenz, EFV, Sustiva ®, Stocrin ® | nnRTI |
| efavirenz + emtricitabine + tenofovir DF, Atripla ® | nnRTI + nRTI |
| emtricitabine, FTC, Emtriva ® | nRTI |
| emtricitabine + tenofovir DF, Truvada ® | nRTI |
| emvirine, Coactinon ® | nnRTI |
| enfuvirtide, Fuzeon ® | FI |
| enteric coated didanosine, Videx EC ® | nRTI |
| etravirine, TMC-125 | nnRTI |
| fosamprenavir calcium, Lexiva ® | PI |
| indinavir, Crixivan ® | PI |
| lamivudine, 3TC, Epivir ® | nRTI |
| lamivudine + zidovudine, Combivir ® | nRTI |
| lopinavir | PI |
| lopinavir + ritonavir, Kaletra ® | PI |
| maraviroc, Selzentry ® | EI |
| nelfinavir, Viracept ® | PI |
| nevirapine, NVP, Viramune ® | nnRTI |
| raltegravir, MK-0518, Isentress ® | InI |
| rilpivirine, TMC-278 | nnRTI |
| ritonavir, Norvir ® | PI |
| saquinavir, Invirase ®, Fortovase ® | PI |
| stavudine, d4T, didehydrodeoxythymidine, Zerit ® | nRTI |
| tenofovir DF (DF = disoproxil fumarate), TDF, Viread ® | nRTI |
| tipranavir, Aptivus ® | PI |

EI = entry inhibitor;
FI = fusion inhibitor;
InI = integrase inhibitor;
PI = protease inhibitor;
nRTI = nucleoside reverse transcriptase inhibitor;
nnRTI = non-nucleoside reverse transcriptase inhibitor.
Some of the drugs listed in the table are used in a salt form; e.g., abacavir sulfate, indinavir sulfate, atazanavir sulfate, nelfinavir mesylate.

It is understood that the scope of combinations of the compounds of this invention with anti-HIV agents is not limited to the HIV antivirals listed in Table A, but includes in principle any combination with any pharmaceutical composition useful for the treatment or prophylaxis of AIDS. The HIV antiviral agents and other agents will typically be employed in these combinations in their conventional dosage ranges and regimens as reported in the art, including, for example, the dosages described in the Physicians' Desk Reference, Thomson PDR, Thomson PDR, 57^{th} edition (2003), the 58^{th} edition (2004), the 59^{th} edition (2005), and so forth. The dosage ranges for a compound of the invention in these combinations are the same as those set forth above.

The compounds of this invention are also useful in the preparation and execution of screening assays for antiviral compounds. For example, the compounds of this invention are useful for isolating enzyme mutants, which are excellent screening tools for more powerful antiviral compounds. Furthermore, the compounds of this invention are useful in establishing or determining the binding site of other antivirals to HIV integrase, e.g., by competitive inhibition. Thus the compounds of this invention are commercial products to be sold for these purposes.

Abbreviations employed herein include the following: Bn=benzyl; DMF=N,N-dimethylformamide; ES MS=electrospray mass spectroscopy; EtOH=ethanol; HTLV=human T-lymphotropic virus; NMR=nuclear magnetic resonance; THF=tetrahydrofuran; XMRV=xenotropic murine leukemia-related retrovirus.

The compounds of the present invention can be readily prepared according to the following reaction schemes and examples, or modifications thereof, using readily available starting materials, reagents and conventional synthesis procedures. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction schemes and examples. Unless otherwise indicated, all variables are as defined above.

Scheme 1 presents a general method for preparing compounds of Formula I in which X is O. A solution of amidodiester 1-1 in a suitable solvent (e.g., alcohol, THF, diethyl ether, or toluene) is treated with base [e.g., Na alkoxide, K alkoxide, Na or K bis(trimethylsilyl)amide, NaH (see Smith et al., Bioorg. Med. Chem. Lett. 2007, p 673), or K₂CO₃] in the presence of a cyclic polyether (e.g., 18-crown-6; see Micovic et al, J. Chem. Soc., Perkin Trans. 1 1996, p 2041) to afford the Dieckmann cyclization product 1-2, which can be readily isolated as the corresponding sodium or potassium salt. Decarboxylation of 1-2 in the presence of an acid [e.g., aqueous. acetic acid (see Smith et al., Bioorg. Med. Chem. Lett. 2007, p 673) or aqueous sulfuric acid (Ashi et al., Tetrahedron 2005, p. 11107)] provides the enol lactam 1-3, which can be reacted with 2,2-dialkoxy-3-halopropionate 1-4 (e.g., 4-nitrophenyl 3-bromo 2,2-diethoxypropionate; see Cohen et al., Org. Letters, 2007, p 2517) in the presence of a base such as NaH or sodium or potassium bis(trimethylsilyl)amide to afford the dialkoxypyranolactam intermediate 1-5. Acid catalyzed hydrolysis of the ketal moiety provides the pyranolactam 1-6.

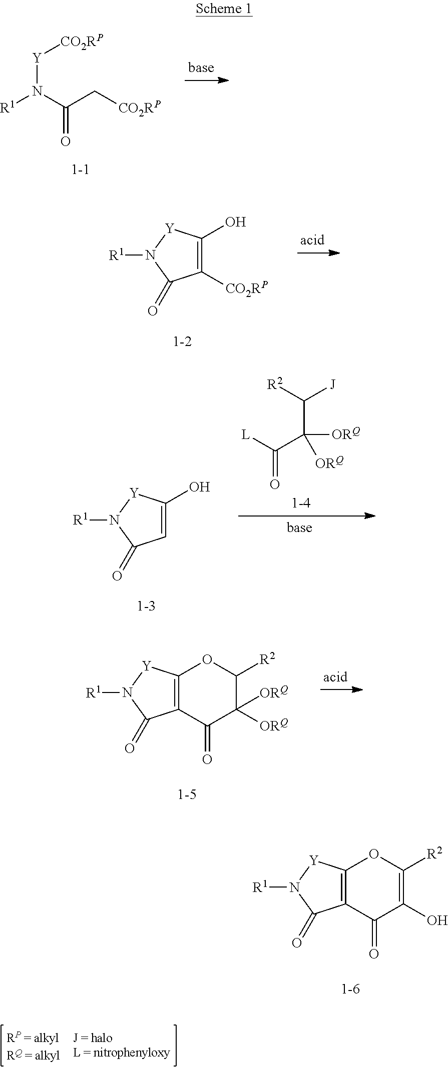

Scheme 2 presents a general method for preparing compounds of Formula I in which X is NR³. The 3-hydroxy group on 1-6 can be protected with a suitable protecting group PG such as benzyl or allyl to afford 2-1, which can be treated with a primary amine in a suitable solvent (e.g., an ether such as THF or an alcohol such as ethanol or methanol) to afford 2-2. Deprotection of the hydroxy group provides the desired dihydropyridolactam 2-3.

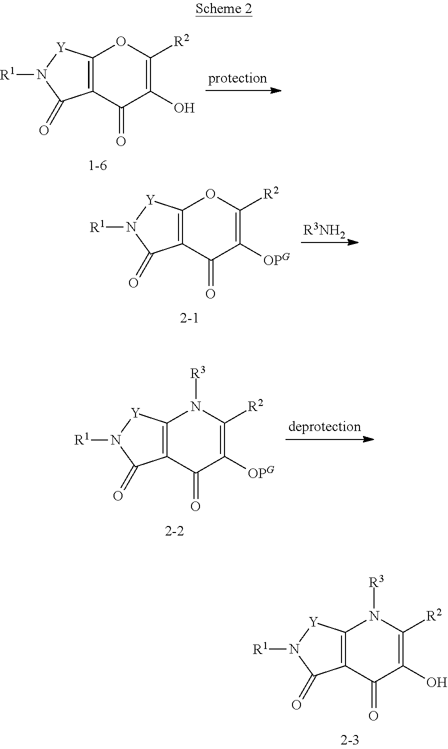

Scheme 3 presents a method for introducing allylic groups and derivatives thereof into the 2-position of the dihydropyridine ring, wherein 2-2a is O-allylated to provide 3-1 which can be heated at 80-120° C. in a suitable organic solvent (e.g., toluene, ethanol, or a combination thereof) to afford the corresponding Claisen rearrangement product 3-2. The olefinic moiety of 3-2 can be subjected to hydroxylation, oxyamination, or cyclopropanation to afford further desired compounds 3-3a, 3-3 b and 3-3c.

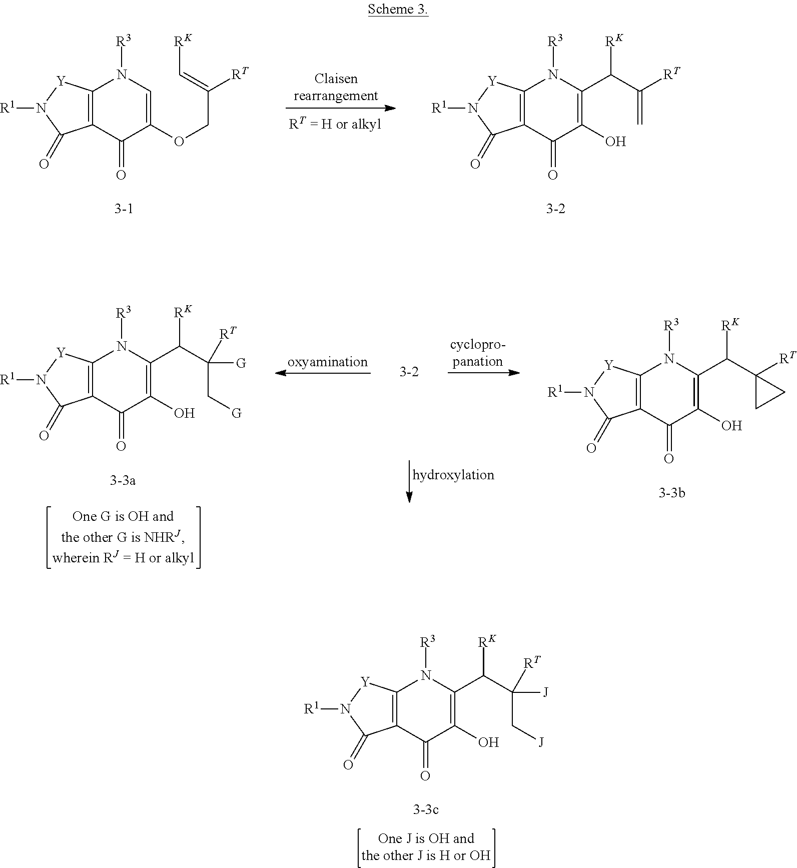

Scheme 4 exemplifies methods suitable for the preparation of tricyclic compounds of Formula Ia, wherein the diolefin 4-1 is subjected to ring-closing metathesis (RCM) chemistry using a suitable catalyst such as Grubbs II catalyst (CAS Registry No. 246047-72-3) to form tricyclic 4-2 (see Deiters & Martin, Chem. Rev. 2004, p 2199). The olefin in 4-2 can be hydrogenated to afford 4-3, or further functionalized by (i) hydroxylation, (ii) hydroxylation followed by O-alkylation to provide alkoxy group(s), (iii) epoxidation followed by alkylation to introduce both alkyl and hydroxy, and (iv) cyclopropanation to afford, after deprotection of the hydroxy group, tricyclics of formula 4-4.

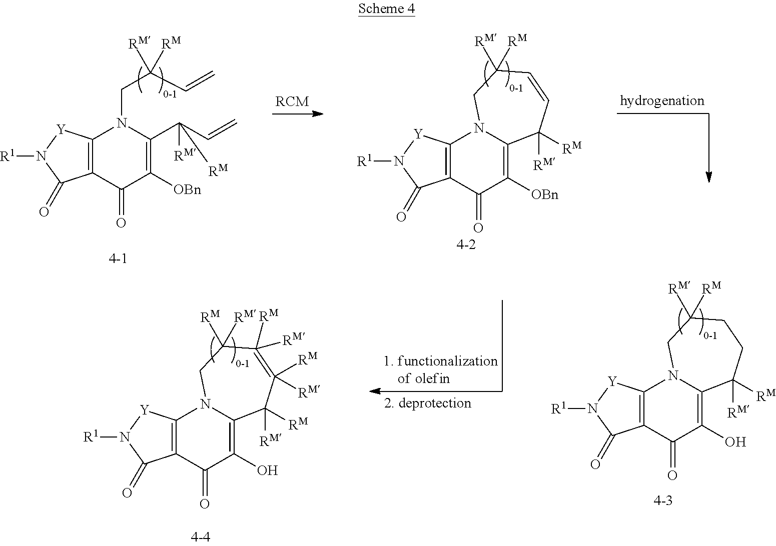

In the methods for preparing compounds of the present invention set forth in the foregoing schemes, functional groups in various moieties and substituents (in addition to those already explicitly noted in the foregoing schemes) may be sensitive or reactive under the reaction conditions employed and/or in the presence of the reagents employed. Such sensitivity/reactivity can interfere with the progress of the desired reaction to reduce the yield of the desired product, or possibly even preclude its formation. Accordingly, it may be necessary or desirable to protect sensitive or reactive groups on any of the molecules concerned. Protection can be achieved by means of conventional protecting groups, such as those described in Protective Groups in Organic Chemistry, ed. J. F. W. McOmie, Plenum Press, 1973 and in T. W. Greene & P. G. M. Wuts, Protective Groups in Organic Synthesis, John Wiley & Sons, 3^{rd} edition, 1999, and 2^{nd} edition, 1991. The protecting groups may be removed at a convenient subsequent stage using methods known in the art. Alternatively the interfering group can be introduced into the molecule subsequent to the reaction step of concern.

The following examples serve only to illustrate the invention and its practice. The examples are not to be construed as limitations on the scope or spirit of the invention. In these examples, “room temperature” refers to a temperature in a range of from about 20° C. to about 25° C.

### Example 1

### 6-(4-Fluorobenzyl)-3-hydroxy-7,8-dihydro-4H-pyrano[3,2-c]pyridine-4,5(6H)-dione

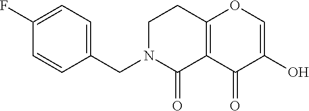

### Step 1: Ethyl 3-[N-(3-ethoxy-3-oxopropyl)-N-(4-fluorobenzyl)]amino-3-oxopropanoate

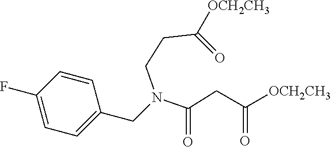

To a solution of 4-fluorobenzylamine (25.4 g, 0.20 mol) in ethanol (200 mL) at room temperature, ethyl acrylate (24.2 mL, 0.22 mol) was added. The reaction mixture was stirred at room temperature overnight and concentrated under vacuum. Residual ethanol and acrylate were removed by co-evaporation with toluene. The crude product (45.7 g, 0.20 mol), used without further purification, diisopropylethylamine (42.4 mL, 0.24 mol), and 4-(N,N-dimethylamino)pyridine (2.5 g, 20 mmol) were dissolved in dichloromethane (300 mL) and cooled to 0° C. The mixture was treated with a solution of ethyl 3-chloro-3-oxopropionate (28.7 mL, 0.22 mol) in chloroform (100 mL) and stirred at 0° C. for one hour. The mixture was allowed to warm up to room temperature and stirred overnight. The reaction mixture was partitioned with water. The organic extract was washed with brine, dried over anhydrous sodium sulfate, filtered, and concentrated under vacuum. The residue was subjected to column chromatography on silica gel eluting with 35% ethyl acetate in hexane. Collection and concentration of appropriate fractions provided title compound as yellow oil. ES MS M+1=340

### Step 2: Ethyl 1-(4-fluorobenzyl)-4-hydroxy-2-oxo-1,2,5,6-tetrahydropyridine-3-carboxylate, sodium salt

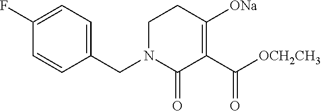

To a solution of sodium ethoxide (3.8 g, 55 mmol) in ethanol (120 mL) at 0° C. under an atmosphere of nitrogen, a solution of ethyl 3-[N-(3-ethoxy-3-oxopropyl)-N-(4-fluorobenzyl)]amino-3-oxopropanoate (17.8 g, 52 mol) in ethanol (50 mL) was added over a period of 15 minutes. The resultant solution was stirred at room temperature for two hours. The reaction mixture was concentrated under vacuum to about 100 mL, and was treated with diethyl ether (25 mL). The white precipitate was collected by filtration, washed with a cold mixture of EtOH and ether (1:2 v/v), and air dried to provide the title compound.

### Step 3: 1-(4-Fluorobenzyl)piperidine-2,4-dione

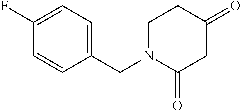

A solution of ethyl 1-(4-fluorobenzyl)-4-hydroxy-2-oxo-1,2,5,6-tetrahydropyridine-3-carboxylate, sodium salt (20.0 g, 63.4 mmol) in 30% aqueous acetic acid (150 mL) was refluxed for 2 hours. The reaction mixture was cooled to room temperature and extracted with methylene chloride (3×). The organic extracts were combined, washed with water, then brine, dried over sodium sulfate, filtered, and concentrated under vacuum. The residue was subjected to column chromatography on a 100 g silica gel column cartridge eluted with 40-100% ethyl acetate:hexane linear gradient mixture at 85 mL/minute over 30 minutes. Collection and concentration of appropriate fractions provided 1-(4-fluorobenzyl)piperidine-2,4-dione. ¹H NMR (400 MHz, CDCl₃): δ 7.26 (dd, J=8.6, 5.2 Hz, 2H), 7.04 (t, J=8.6 Hz, 2H), 4.66 (s, 2H), 3.49 (t, J=6.2 Hz, 2H), 3.42 (s, 2H), 2.59-2.51 (m, 2H). ES MS M+1=222.

### Step 4: 3,3-Diethoxy-6-(4-fluorobenzyl)-7,8-dihydro-2H-pyrano[3,2-c]pyridine-4,5(3H,6H)-dione

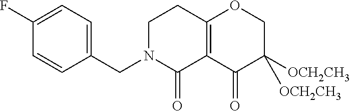

To a suspension of NaH (2.3 g, 56.3 mmol, 60% in mineral oil; washed with hexane) in dry THF (50 mL), a solution of 1-(4-fluorobenzyl)piperidine-2,4-dione (11.3 g, 51.2 mmol) in THF (150 mL) was added dropwise, followed by addition of a solution of 4-nitrophenyl 3-bromo-2,2-diethoxypropanoate (9.27 g, 25.6 mmol; Organic Letters, 9, 2517, 2007) in THF (50 mL). The resulting reaction mixture was refluxed for 4 hours under an atmosphere of nitrogen. The product mixture was cooled, treated with saturated aqueous ammonium chloride (50 mL), and extracted with methylene chloride (3×100 mL). The organic extracts were combined, washed with water, 10% aq. sodium carbonate, and brine, then dried over sodium sulfate, and then concentrated in vacuo. The residue was subjected to column chromatography on a 340 g silica gel cartridge eluted at 100 mL/minute with a 0-10% methanol:chloroform linear gradient mixture over 70 minutes. Collection and concentration of appropriate fractions provided 3,3-diethoxy-6-(4-fluorobenzyl)-7,8-dihydro-2H-pyrano[3,2-c]pyridine-4,5(3H,6H)-dione as foamy solid. ¹H NMR (400 MHz, CDCl₃): δ 7.42-7.10 (m, 2H), 7.03-6.97 (m, 2H), 4.59 (s, 2H), 4.41 (s, 2H), 3.73-3.62 (m, 4H), 3.32 (t, J=6.8 Hz, 2H), 2.58 (t, J=6.7 Hz, 2H), 1.22 (t, J=7.1 Hz, 6H). ES MS M+1=364

### Step 5: 6-(4-Fluorobenzyl)-3-hydroxy-7,8-dihydro-4H-pyrano[3,2-c]pyridine-4,5(6H)-dione

A solution of 3,3-diethoxy-6-(4-fluorobenzyl)-7,8-dihydro-2H-pyrano[3,2-c]pyridine-4,5(3H,6H)-dione (2.86 g, 7.87 mmol) in a mixture of THF (30 mL), TFA (15 mL), and water (3 mL) was refluxed for 1 hour. The product mixture was concentrated under vacuum. The residue was subjected to column chromatography on silica gel eluted at 30 mL/minute with a 0-10% methanol:chloroform linear gradient mixture over 15 minutes. Collection and concentration of appropriate fractions provided 6-(4-fluorobenzyl)-3-hydroxy-7,8-dihydro-4H-pyrano[3,2-c]pyridine-4,5(6H)-dione. ¹H NMR (400 MHz, CDCl₃): δ 7.79 (s, 1H), 7.29 (dd, J=8.4, 5.3 Hz, 2H), 7.05-6.97 (m, 2H), 4.66 (s, 2H), 3.51-3.43 (m, 2H), 2.92-2.85 (m, 2H). Exact mass M+1=290.0827

### Example 2

### 2-Allyl-6-(4-fluorobenzyl)-3-hydroxy-1-methyl-7,8-dihydro-1,6-naphthyridine-4,5(1H,6H)-dione

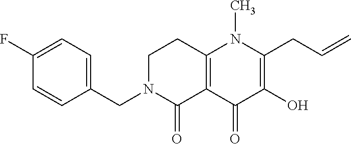

### Step 1: 3-(Allyloxy)-6-(4-fluorobenzyl)-7,8-dihydro-4H-pyrano[3,2-c]pyridine-4,5(6H)-dione

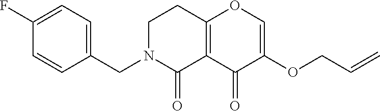

A mixture of 6-(4-fluorobenzyl)-3-hydroxy-7,8-dihydro-4H-pyrano[3,2-c]pyridine-4,5(6H)-dione (560 mg, 1.94 mmol; Example 1), cesium carbonate (883 mg, 2.71 mmol), and allyl bromide (201 μt, 2.04 mmol) in DMF (5 mL) was stirred at room temperature overnight. The reaction mixture was diluted with ethyl acetate and washed with water and then brine. The organic extract was dried over MgSO₄, filtered, and concentrated in vacuo. The residue was subjected to column chromatography on a 12 g silica gel column cartridge eluted at 30 mL/minute with a 0-10% methanol:chloroform linear gradient mixture over 15 minutes. Collection and concentration of appropriate fractions provided 3-(allyloxy)-6-(4-fluorobenzyl)-7,8-dihydro-4H-pyrano[3,2-c]pyridine-4,5(6H)-dione. ¹H NMR (400 MHz, CDCl₃): δ 7.51 (s, 1 H), 7.30 (dd, J=8.4, 5.4 Hz, 2H), 7.01 (dd, J=8.6, 8.5 Hz, 2H), 5.98 (ddt, J=17.3, 10.4, 5.7 Hz, 1H), 5.36 (dd, J=17.3, 1.5 Hz, 1H), 5.30 (dd, J=10.4, 1.5 Hz, 1H), 4.65 (s, 2H), 4.55 (dt, J=5.7, 1.4 Hz, 2H), 3.43 (t, J=6.8 Hz, 2H), 2.80 (t, J=6.8 Hz, 2H). ES MS M+1=330.

### Step 2: 3-Allyloxy-6-(4-fluorobenzyl)-3-hydroxy-1-methyl-7,8-dihydro-1,6-naphthyridine-4,5(1H,6H)-dione

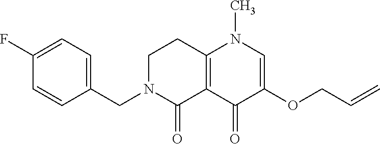

A solution of 3-(allyloxy)-6-(4-fluorobenzyl)-7,8-dihydro-4H-pyrano[3,2-c]pyridine-4,5(6H)-dione (50 mg, 0.15 mmol) in THF (200 μL) was treated with methylamine (152 μt, 0.30 mmol, 2M solution in THF) was stirred at room temperature for 3 days. The reaction mixture was concentrated in vacuo. The residue was subjected to column chromatography on a 12 g silica gel column cartridge eluted at 30 mL/minute with a 0-6% methanol:chloroform linear gradient mixture over 15 minutes, then a 90:10:1 chloroform:methanol:conc. ammonium hydroxide mixture for 5 minutes. Collection and concentration of appropriate fractions provided 3-allyloxy-6-(4-fluorobenzyl)-3-hydroxy-1-methyl-7,8-dihydro-1,6-naphthyridine-4,5(1H,6H)-dione. ¹H NMR (400 MHz, CDCl₃): δ 7.30 (dd, J=8.2, 5.2 Hz, 2H), 6.98 (t, J=8.5 Hz, 2H), 6.89 (s, 1H), 5.98 (ddt, J=17.3, 10.4, 5.6 Hz, 1H), 5.36 (dd, J=17.3, 1.5 Hz, 1H), 5.23 (d, J=10.5 Hz, 1H), 4.61 (s, 2H), 4.56 (d, J=5.6 Hz, 2H), 3.57 (s, 3H), 3.34 (t, J=6.4 Hz, 2H), 2.75 (t, J=6.5 Hz, 2H). ES MS M+1=343.

### Step 3: 2-Allyl-6-(4-fluorobenzyl)-3-hydroxy-1-methyl-7,8-dihydro-1,6-naphthyridine-4,5(1H,6H)-dione

A solution of 3-allyloxy-6-(4-fluorobenzyl)-3-hydroxy-1-methyl-7,8-dihydro-1,6-naphthyridine-4,5(1H,6H)-dione (20 mg, 0.058 mmol) in ethanol (0.4 mL) and toluene (0.4 mL) was heated in a sealed tube at 100° C. for 2 days. The reaction mixture was concentrated in vacuo and the residue subjected to reverse phase chromatography on a C-18 column eluting with a 3-90% acetonitrile:water (with 0.1% trifluoroacetic acid) linear gradient mixture over 30 minutes at 30 mL/minute. Collection and lyophilization of appropriate fractions provided 2-allyl-6-(4-fluorobenzyl)-3-hydroxy-1-methyl-7,8-dihydro-1,6-naphthyridine-4,5(1H,6H)-dione. ¹H NMR (400 MHz, CDCl₃): δ 7.37-7.31 (m, 2H), 7.06 (t, J=8.5 Hz, 2H), 5.88 (ddt, J=17.3, 10.2, 5.7 Hz, 1H), 5.27 (d, J=10.2 Hz, 1H), 5.10 (d, J=17.3 Hz, 1H), 4.72 (s, 2H), 3.89 (s, 2 H), 3.76 (s, 3H), 3.66 (t, J=6.8 Hz, 1H), 3.28 (t, J=6.8 Hz, 1H). Exact mass M+1=343.1447

### Example 3

### 1-But-3-en-1-yl-6-(4-fluorobenzyl)-3-hydroxy-2-(1-methylprop-2-en-1-yl)-7,8-dihydro-1,6-napthyridine-4,5(1H,6H)-dione

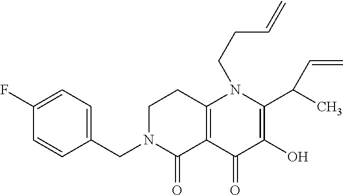

### Step 1: 3-[(2E)-But-2-en-1-yloxy]-6-(4-fluorobenzyl)-7,8-dihydro-4H-pyrano[3,2-c]pyridine-4,5(6H)-dione

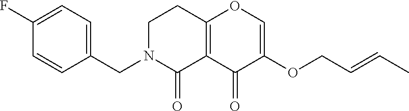

A mixture of 6-(4-fluorobenzyl)-3-hydroxy-7,8-dihydro-4H-pyrano[3,2-c]pyridine-4,5(6H)-dione (200 mg, 0.691 mmol; Example 1), cesium carbonate (293 mg, 0.899 mmol), and crotyl bromide (85 μL, 830 mmol) in DMF (5 mL) was stirred the reaction mixture overnight. The reaction mixture was diluted with ethyl acetate and washed with water and then brine. The organic extract was dried over MgSO₄, filtered, and concentrated in vacuo. The residue was subjected to column chromatography on a 12 g silica gel column cartridge eluted at 30 mL/minute with a 0-10% methanol:chloroform linear gradient mixture over 15 minutes. Collection and concentration of appropriate fractions provided title compound. ¹H NMR (400 MHz, CDCl₃): δ 7.49 (s, 1H), 7.30 (dd, J=8.4, 5.4 Hz, 2H), 7.01 (t, J=8.5 Hz, 2H), 5.85-5.76 (m, 1H), 5.70-5.62 (m, 1H), 4.65 (s, 2H), 4.45 (dt, J=6.3, 1.2 Hz, 2H), 3.42 (t, J=6.8 Hz, 2H), 2.79 (t, J=6.8 Hz, 2H), 1.73 (dq, J=6.4, 1.3 Hz, 3H). ES MS M+1=344

### Step 2: 1-(But-3-en-1-yl)-3-[(2E)-but-2-en-1-yloxy]-6-(4-fluorobenzyl)-7,8-dihydro-1,6-naphthyridine-4,5(1H,6H)-dione

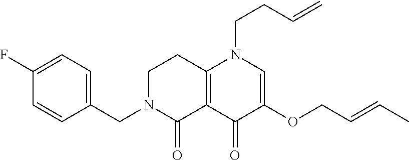

A solution of 3-[(2E)-but-2-en-1-yloxy]-6-(4-fluorobenzyl)-7,8-dihydro-4H-pyrano[3,2-c]pyridine-4,5(6H)-dione (168 mg, 0.489 mmol) and 3-butenylamine (39 mg, 0.489 mmol) in THF (1 mL) was stirred at room temperature for 4 hours. The reaction mixture was concentrated in vacuo and the residue was subjected to column chromatography on a 12 g silica gel column cartridge eluted with a 0-6% methanol:chloroform mixture linear gradient over 15 minutes at 30 mL/minute, then with a mixture of 90:10:1 chloroform:methanol:concentrated NH₄OH for 5 minutes. Collection and concentration of appropriate fractions provided title compound. ¹H NMR (400 MHz, CDCl₃): δ 7.32 (dd, J=8.3, 5.4 Hz, 2H), 6.99 (t, J=8.5 Hz, 2H), 6.89 (s, 1H), 5.84-5.74 (m, 1H), 5.74-5.61 (m, 2H), 5.14 (d, J=10.2 Hz, 1H), 5.10 (dq, J=17.1, 1.1 Hz, 1H), 4.67 (s, 2H), 4.56 (d, J=6.1 Hz, 2H), 3.82 (t, J=7.1 Hz, 2H), 3.49 (s, 2H), 3.36 (t, J=6.3 Hz, 3H), 2.73 (t, J=6.3 Hz, 2H), 2.43 (q, J=7.1 Hz, 2H), 1.72 (br d, J=6.2 Hz, 31:1). ES MS M+1=397

### Step 3: 1-(But-3-en-1-yl)-2-(but-3-en-2-yl)-6-(4-fluorobenzyl)-3-hydroxy-7,8-dihydro-1,6-naphthyridine-4,5(1H,6H)-dione

A solution of 1-(but-3-en-1-yl)-3-[(2E)-but-2-en-1-yloxy]-6-(4-fluorobenzyl)-7,8-dihydro-1,6-naphthyridine-4,5(1H,6H)-dione (155 mg, 0.391 mmol) in ethanol (3 mL) was heated in a sealed tube at 100° C. for 7 days. The reaction mixture was concentrated in vacuo and the residue subjected to reverse phase chromatography on a C-18 column eluting with a 3-95% acetonitrile:water (with 0.1% trifluoroacetic acid) mixture linear gradient over 30 minutes at 30 mL/minute. Collection and lyophilization of appropriate fractions provided 1-(but-3-en-1-yl)-2-(but-3-en-2-yl)-6-(4-fluorobenzyl)-3-hydroxy-7,8-dihydro-1,6-naphthyridine-4,5(1H,6H)-dione. ¹H NMR (400 MHz, CDCl₃): δ 8.64 (br s, 1H), 7.30 (dd, J=8.3, 5.3 Hz, 2H), 7.05 (t, J=8.4 Hz, 2H), 6.31-6.20 (m, 1H), 5.75 (ddt, J=17.0, 10.4, 6.8 Hz, 1H), 5.28-5.10 (m, 4H), 4.79-4.60 (m, 2H), 4.49-4.30 (m, 2H), 4.19-4.07 (m, 1H), 3.74-3.64 (m, 2H), 2.53 (q, J=7.6 Hz, 2H), 1.60 (d, J=7.0 Hz, 3H). Exact mass M+1=397.1942

### Example 4

### 3-(4-Fluorobenzyl)-6-hydroxy-7-methyl-2,3,8,9,10,11-hexahydroazepino[1,2-a]-1,6-napthyridine-4,5(1H,7H)-dione

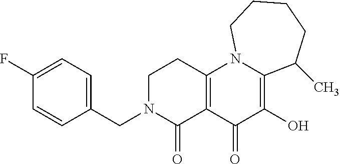

### Step 1: 3-(Benzyloxy)-1-(but-3-en-1-yl)-2-(but-3-en-2-yl)-6-(4-fluorobenzyl)-7,8-dihydro-1,6-naphthyridine-4,5(1H,6H)-dione

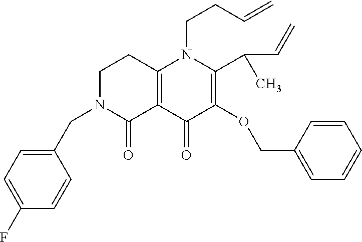

A mixture of 1-(but-3-en-1-yl)-2-(but-3-en-2-yl)-6-(4-fluorobenzyl)-3-hydroxy-7,8-dihydro-1,6-naphthyridine-4,5(1H,6H)-dione (30 mg, 76 μmol), cesium carbonate (37 mg, 0.114 mmol), and benzyl bromide (9 μL, 76 umol) in DMF (0.5 mL) was stirred at room temperature for 2 hours. The reaction mixture was diluted with ethyl acetate and washed with water and then brine. The organic extract was dried over MgSO₄, filtered, and concentrated in vacuo. The residue was subjected to column chromatography on a 12 g silica gel column cartridge eluted at 30 mL/minute with a 0-10% methanol:chloroform mixture linear gradient over 15 minutes. Collection and concentration of appropriate fractions provided title compound. ¹H NMR (400 MHz, CDCl₃): δ 7.48 (d, J=7.2 Hz, 2H), 7.37-7.20 (m, 5H), 7.00 (t, 8.7 Hz, 2H), 5.31 (d, J=11.1 Hz, 1H), 5.23 (d, J=11.1 Hz, 1H), 5.19-4.97 (m), 5.11 (s, 2H), 4.68 (s, 2H), 3.84 (t, J=7.7 Hz, 2H), 3.38 (t, J=6.3 Hz, 2H), 2.82 (t, J=6.2 Hz, 2H), 2.40-2.17 (m), 1.29 (d, J=7.1 Hz, 2H). ES MS M+1=487

### Step 2: 6-(Benzyloxy)-3-(4-fluorobenzyl)-7-methyl-2,3,10,11-tetrahydroazepino[1,2-a][1,6]naphthyridine-4,5(1H,7H)-dione

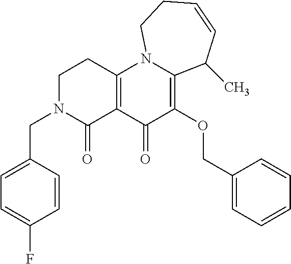

A solution of 3-(benzyloxy)-1-(but-3-en-1-yl)-2-(but-3-en-2-yl)-6-(4-fluorobenzyl)-7,8-dihydro-1,6-naphthyridine-4,5(1H,6H)-dione (18 mg, 37 μmol) and Grubbs II catalyst (6.3 mg, 7.4 μmol; CAS #246047-72-3) in CH₂Cl₂ (3.7 mL) was stirred under an atmosphere of nitrogen at room temperature overnight. The reaction mixture was concentrated in vacuo and the residue was subjected to column chromatography on a 12 g silica gel column cartridge eluted with a 0-10% methanol:chloroform mixture with 1% concentrated NH₄OH linear gradient over 15 minutes at 30 mL/minute. Collection and concentration of appropriate fractions provided 6-(benzyloxy)-3-(4-fluorobenzyl)-7-methyl-2,3,10,11-tetrahydroazepino[1,2-a][1,6]naphthyridine-4,5(1H,7H)-dione. ¹H NMR (400 MHz, CDCl₃): δ 7.47 (d, J=7.2 Hz, 2H), 7.35-7.29 (m, 5H), 7.00 (t, J=8.4 Hz, 2H), 5.61-5.55 (m, 2H), 5.27 (d, J=11.3 Hz, 1H), 5.22 (d, J=11.2 Hz, 1H), 4.74 (d, J=14.7 Hz, 1H), 4.61 (d, J=14.7 Hz, 1H), 4.36 (p, J=7.4 Hz, 1H), 4.33-4.21 (m, 1H), 4.04 (br d, 1H), 3.47-3.34 (m, 1H), 3.37-3.28 (m, 1H), 2.91-2.84 (m, 1H), 2.67 (dt, J=16.0, 4.8 Hz, 1H), 2.41-2.29 (br signals, 2H), 1.29 (d, J=7.5 Hz, 3H). ES MS M+1=459.

### Step 3: 3-(4-Fluorobenzyl)-6-hydroxy-7-methyl-2,3,8,9,10,11-hexahydroazepino[1,2-c]-1,6-napthyridine-4,5(1H,7H)-dione

A solution of 6-(benzyloxy)-3-(4-fluorobenzyl)-7-methyl-2,3,10,11-tetrahydroazepino[1,2-a][1,6]naphthyridine-4,5(1H,7H)-dione (6 mg, 13 tμmol) in methanol (0.5 mL) at room temperature was hydrogenated over 10% Pd/C (1 mg) at 14 psi for 5 hours. The reaction mixture was filtered, and filtrate was concentrated in vacuo to provide 3-(4-fluorobenzyl)-6-hydroxy-7-methyl-2,3,8,9,10,11-hexahydroazepino[1,2-a]-1,6-napthyridine-4,5(1H,7H)-dione. ¹H NMR (400 MHz, CDCl₃): δ 7.31 (dd, J=8.1, 5.3 Hz, 2H), 6.99 (t, J=8.4 Hz, 2H), 4.76 (d, J=14.7 Hz, 1H), 4.59 (d, J=14.7 Hz, 1H), 4.08 (dt, 15.4, 3.5 Hz, 1H), 4.04-3.85 (m, 2H), 3.43 (dd, J=12.2, 4.3 Hz, 1H), 3.37 (dd, J=14.2, 5.0 Hz, 1H), 3.33 (dt, J=12.7, 5.3 Hz, 1H), 2.96-2.84 (m, 1H), 2.72 (dt, J=15.9, 4.7 Hz, 1H), 2.08-1.96 (m, 1H), 1.93-1.79 (m, 1H), 1.68-1.54 (m, 3H), 1.38 (d, J=7.5 Hz, 3H). Exact mass M+1=371.1761.

### Example 5

### HIV Integrase Assay

### Strand Transfer Catalyzed by Recombinant Integrase

Assays for the strand transfer activity of HIV-1 integrase were conducted in accordance with WO 02/30930 for recombinant integrase. Representative compounds of the present invention exhibit inhibition of strand transfer activity in this assay. For example, the compounds prepared in Examples 1 to 4 were tested in the integrase assay and found to have the IC₅₀ values in Table B.

TABLE B

| Compound | IC₅₀ (μM) |
|---|---|
| 1 | 3.20 |
| 2 | 0.10 |
| 3 | 0.10 |
| 4 | 0.12 |

Further description on conducting the assay using preassembled complexes is found in Wolfe, A. L. et al., J. Virol. 1996, 70: 1424-1432, Hazuda et al., J. Virol. 1997, 71: 7005-7011; Hazuda et al., Drug Design and Discovery 1997, 15: 17-24; and Hazuda et al., Science 2000, 287: 646-650.

### Example 6

### Assay for Inhibition of HIV Replication

An assay for measuring the inhibition of acute HIV infection with HeLa P4-2 cells in a single cycle infectivity assay (alternatively referred to as the “vertical” assay) was conducted in accordance with Joyce, J. G., et al., J. Biol. Chem. 2002, 277, 45811, Hazuda, D. J. et al., Science 2000, 287, 646, and Kimpton, J. et al, J. Virol. 1992, 66, 2232. Infectious virus was produced by transfecting 293T cells with HIV proviral DNA in which the integrase gene was derived from a IIIB isolate and the remainder of the HIV genome was derived from the NL4-3 isolate. Representative compounds of the present invention exhibit inhibition of HIV replication in this assay. For example, the compounds of Examples 1 to 4 were tested in this assay and found to have the IC₅₀ values in Table C.

TABLE C

| Compound | IC₅₀ (μM) |
|---|---|
| 1 | 43 |
| 2 | 5 |
| 3 | 1 |
| 4 | 5 |

### Example 7

### Cytotoxicity

Cytotoxicity was determined by microscopic examination of the cells in each well in the spread assay, wherein a trained analyst observed each culture for any of the following morphological changes as compared to the control cultures: pH imbalance, cell abnormality, cytostatic, cytopathic, or crystallization (i.e., the compound is not soluble or forms crystals in the well). The toxicity value assigned to a given compound is the lowest concentration of the compound at which one of the above changes is observed. Representative compounds of the present invention that were tested in the vertical assay (see Example 6) were examined for cytotoxicity up to a concentration of 0.5 micromolar, and no cytotoxicity was exhibited. In particular, the compounds set forth in Examples 1 to 4 exhibited no cytotoxicity at concentrations up to 50 micromolar.

While the foregoing specification teaches the principles of the present invention, with examples provided for the purpose of illustration, the practice of the invention encompasses all of the usual variations, adaptations and/or modifications that come within the scope of the following claims.

1. A compound of Formula I, or a pharmaceutically acceptable salt thereof: 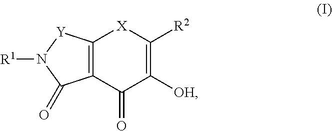
wherein:
X is O or N(R³);
Y is CH(R⁴) or CH(R⁴)CH(R⁴);
R¹ is C₁₋₆ alkyl substituted with R^{J}, wherein R^{J} is AryA or HetA;
R² is H, C₁₋₆ alkyl, or CH(R^{K})R^{L};
R^{K} is H or C₁₋₆ alkyl;
R^{L} is C(R^{T})═CH₂, 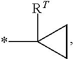
or C(R^{T})R^{C}—CHR^{C};
R^{T} is H or C₁₋₆ alkyl;
one R^{C} is OH and the other R^{C} is H or OH; or one R^{C} is OH and the other R^{C} is NH₂ or NH—C₁₋₆ alkyl;
R³ is H, C₁₋₆ alkyl, or CH(R^{Q})—C₁₋₆ alkenyl;
R^{Q} is H or C₁₋₆ alkyl;
each R⁴ is independently H or C₁₋₆ alkyl;
alternatively, when X is N(R³), R² and R³ together with the atoms to which they are attached form an azacycloalkyl ring such that the compound of Formula I is a compound of Formula Ia: 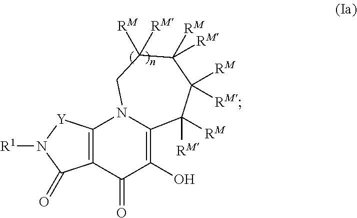
each R^{M} is independently:
(1) H,
(2) C₁₋₆ alkyl,
(3) OH,
(4) C₁₋₆ alkoxy,
(5) oxo which is formed together with the R^{M′} attached to the same carbon, or
(6) methylene which is formed together with an R^{M} on an adjacent ring carbon to provide fused cyclopropyl;
for each R^{M} which is other than oxo, R^{M′} is independently H or C₁₋₆ alkyl;
n is zero or 1;
AryA is an aryl which is optionally substituted with a total of from 1 to 5 substituents, wherein:
(i) from zero to 5 substituents are each independently:
(1) C₁₋₆ alkyl,
(2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, N(R^{A})C(O)N(R^{A})R^{B}, or N(R^{A})C(O)C(O)N(R^{A})R^{B},
(3) O—C₁₋₆ alkyl,
(4) C₁₋₆ haloalkyl,
(5) O—C₁₋₆ haloalkyl,
(6) OH,
(7) halogen,
(8) CN,
(9) NO₂,
(10) N(R^{A})R^{B},
(11) C(O)N(R^{A})R^{B},
(12) C(O)R^{A},
(13) C(O)—C₁₋₆ haloalkyl,
(14) C(O)OR^{A},
(15) OC(O)N(R^{A})R^{B},
(16) SR^{A},
(17) S(O)R^{A},
(18) SO₂R^{A},
(19) SO₂N(R^{A})R^{B},
(20) N(R^{A})SO₂R^{B},
(21) N(R^{A})SO₂N(R^{A})R^{B},
(22) N(R^{A})C(O)R^{B},
(23) N(R^{A})C(O)N(R^{A})R^{B},
(24) N(R^{A})C(O)C(O)N(R^{A})R^{B}, or
(25) N(R^{A})CO₂R^{B}, and
(ii) from zero to 2 substituents are each independently:
(1) CycD,
(2) AryD,
(3) HetD,
(4) HetZ,
(5) C₁₋₆ alkyl substituted with CycD, AryD, HetD, or HetZ, or
(6) C(O)-HetZ or C(O)C(O)-HetZ;
HetA is a heteroaryl which is optionally substituted with a total of from 1 to 5 substituents, wherein:
(i) from zero to 5 substituents are each independently:
(1) C₁₋₆ alkyl,
(2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, N(R^{A})C(O)N(R^{A})R^{B}, or N(R^{A})C(O)C(O)N(R^{A})R^{B},
(3) O—C₁₋₆ alkyl,
(4) C₁₋₆ haloalkyl,
(5) O—C₁₋₆ haloalkyl,
(6) OH,
(7) halogen,
(8) CN,
(9) NO₂
(10) N(R^{A})R^{B},
(11) C(O)N(R^{A})R^{B},
(12) C(O)R^{A},
(13) C(O)—C₁₋₆ haloalkyl,
(14) C(O)OR^{A},
(15) OC(O)N(R^{A})R^{B},
(16) SR^{A},
(17) S(O)R^{A},
(18) SO₂R^{A},
(19) SO₂N(R^{A})R^{B},
(20) N(R^{A})SO₂R^{B},
(21) N(R^{A})SO₂N(R^{A})R^{B},
(22) N(R^{A})C(O)R^{B},
(23) N(R^{A})C(O)N(R^{A})R^{B},
(24) N(R^{A})C(O)C(O)N(R^{A})R^{B}, or
(25) N(R^{A})CO₂R^{B}, and
(ii) from zero to 2 substituents are each independently:
(1) CycD,
(2) AryD,
(3) HetD,
(4) HetZ,
(5) C₁₋₆ alkyl substituted with CycD, AryD, HetD, or HetZ, or
(6) C(O)-HetZ or C(O)C(O)-HetZ;
each CycD is independently a C₃₋₈ cycloalkyl which is optionally substituted with from 1 to 4 substituents each of which is independently halogen, C₁₋₆ alkyl, OH, O—C₁₋₆ alkyl, or C₁₋₆ haloalkyl;
each AryD is independently phenyl or naphthyl, wherein the phenyl or naphthyl is optionally substituted with from 1 to 5 substituents each of which is independently any one of the substituents (1) to (25) as set forth above in part (i) of the definition of AryA;
each HetD is independently a 5- or 6-membered heteroaromatic ring containing from 1 to 4 heteroatoms independently selected from N, O and S, wherein each N is optionally in the form of an oxide, and wherein the heteroaromatic ring is optionally substituted with from 1 to 4 substituents each of which is independently any one of the substituents (1) to (25) as set forth above in part (i) of the definition of HetA;
each HetZ is independently a 4- to 7-membered, saturated or mono-unsaturated heterocyclic ring containing at least one carbon atom and from 1 to 4 heteroatoms independently selected from N, O and S, where each S is optionally oxidized to S(O) or S(O)₂, wherein the saturated or mono-unsaturated heterocyclic ring is optionally substituted with from 1 to 4 substituents each of which is independently halogen, C₁₋₆ alkyl, C₁₋₆ haloalkyl, O—C₁₋₆ alkyl, O—C₁₋₆ haloalkyl, oxo, C(O)N(R^{A})R^{B}, C(O)C(O)N(R^{A})R^{B}, C(O)R^{A}, CO₂R^{A}, SR^{A}, S(O)R^{A}, SO₂R^{A}, or SO₂N(R^{A})R^{B};
each R^{A} is independently H or C₁₋₆ alkyl;
each R^{B} is independently H or C₁₋₆ alkyl;
each aryl is independently (i) phenyl, (ii) a 9- or 10-membered bicyclic, fused carbocyclic ring system in which at least one ring is aromatic, or (iii) an 11- to 14-membered tricyclic, fused carbocyclic ring system in which at least one ring is aromatic; and
each heteroaryl is independently (i) a 5- or 6-membered heteroaromatic ring containing from 1 to 4 heteroatoms independently selected from N, O and S, wherein each N is optionally in the form of an oxide, (ii) a 9- or 10-membered bicyclic, fused ring system, or (iii) an 11- to 14-membered tricyclic, fused ring system, wherein the fused ring system in (ii) or (iii) contains from 1 to 4 heteroatoms independently selected from N, O and S, and wherein in the fused ring system of (ii) or (iii) any one or more of the rings contain one or more of the heteroatoms, at least one ring is aromatic, each N in a ring is optionally in the form of an oxide, and each S is optionally S(O) or S(O)₂.

2. A compound according to claim 1, or a pharmaceutically acceptable salt thereof, wherein X is N(R³).

3. A compound according to claim 1, or a pharmaceutically acceptable salt thereof, wherein X is O.

4. A compound according to claim 1, or a pharmaceutically acceptable salt thereof, wherein Y is CH₂, CH(CH₃), CH₂CH₂, CH(CH₃)CH(CH₃), CH₂CH(CH₃), CH(CH₃)CH₂, or CH₂CH₂.

5. A compound according to claim 4, or a pharmaceutically acceptable salt thereof, wherein Y is CH₂CH₂.

6. A compound according to claim 1, or a pharmaceutically acceptable salt thereof, wherein R¹ is: 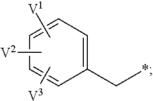
the asterisk * denotes the point of attachment of R¹ to the rest of the compound;
V¹ and V² are each independently:
(1) H,
(2) C₁₋₄ alkyl,
(3) OH,
(4) O—C₁₋₄ alkyl,
(5) C₁₋₄ haloalkyl,
(6) O—C₁₋₄ haloalkyl,
(7) halogen,
(8) CN,
(9) N(R^{A})R^{B},
(10) C(O)N(R^{A})R^{B},
(11) C(O)R^{A},
(12) C(O)OR^{A},
(13) SR^{A},
(14) S(O)R^{A},
(15) SO₂R^{A},
(16) N(R^{A})SO₂R^{B},
(17) N(R^{A})SO₂N(R^{A})R^{B},
(18) N(R^{A})C(O)R^{B},
(19) N(R^{A})C(O)C(O)N(R^{A})R^{B},
(20) HetD,
(21) HetZ, or
(22) C(O)-HetZ, wherein:
HetD is a 5- or 6-membered heteroaromatic ring containing a total of from 1 to 3 heteroatoms independently selected from 1 to 3 N atoms, from zero to 1 O atom, and from zero to 1 S atom, wherein the heteroaromatic ring is optionally substituted with from 1 to 3 substituents each of which is independently C₁₋₄ alkyl, OH, O—C₁₋₄ alkyl, halogen, CN, C(O)N(R^{A})R^{B}, C(O)R^{A}, C(O)OR^{A}, or SO₂R^{A},
HetZ is a 5- or 6-membered saturated heterocyclic ring containing a total of from 1 to 2 heteroatoms selected from 1 to 2 N atoms, zero to 1 O atom, and zero to 1 S atom, wherein the S atom is optionally S(O) or SO₂, wherein the saturated heterocyclic ring is optionally substituted with from 1 to 2 substituents each of which is independently C₁₋₄ alkyl, oxo, C(O)N(R^{A})R^{B}, C(O)R^{A}, CO₂R^{A}, or SO₂R^{A},
and with the proviso that when HetZ is attached to the rest of the compound via the C(O) moiety, then HetZ is attached to the C(O) via a ring N atom;
or alternatively V¹ and V² are respectively located on adjacent carbons in the phenyl ring and together form methylenedioxy or ethylenedioxy;
V³ is:
(1) H,
(2) C₁₋₄ alkyl,
(3) O—C₁₋₄ alkyl,
(4) C₁₋₄ haloalkyl,
(5) O—C₁₋₄ haloalkyl, or
(6) halogen;
each R^{A} is independently H or C₁₋₄ alkyl; and
each R^{B} is independently H or C₁₋₄ alkyl.

7. A compound according to claim 6, or a pharmaceutically acceptable salt thereof, wherein V¹ and V² are each independently:
(1) H,
(2) CH₃,
(3) CF₃,
(4) OH,
(5) OCH₃,
(6) Cl, Br, or F,
(7) CN,
(8) C(O)NH₂,
(9) C(O)NH(CH₃),
(10) C(O)N(CH₃)₂, or
(11) SO₂CH₃; and
V³ is H, Cl, Br, F, CH₃, or OCH₃.

8. A compound according to claim 6, or a pharmaceutically acceptable salt thereof, wherein R¹ is 4-fluorobenzyl, 3-chloro-4-fluorobenzyl, or 4-fluoro-3-methylbenzyl.

9. A compound according to claim 1, or a pharmaceutically acceptable salt thereof, wherein:
R² is H, C₁₋₄ alkyl, or CH(R^{K})R^{L};
R^{K} is H or C₁₋₄ alkyl;
R^{L} is C(R^{T})═CH₂, 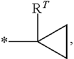
1-hydroxyethyl, 2-hydroxyethyl, 1,2-dihydroxyethyl, 2-amino-1-hydroxyethyl, or 1-amino-2-hydroxyethyl;
R^{T} is H or C₁₋₄ alkyl;
R³ is H, C₁₋₄ alkyl, or CH(R^{Q})—C₁₋₄ alkenyl;
R^{Q} is H or C₁₋₄ alkyl;
each R⁴ is independently H or C₁₋₄ alkyl;
alternatively, when X is N(R³) and R² and R³ together with the atoms to which they are attached form an azacycloalkyl ring of Formula Ia, then each R^{M} is independently H, C₁₋₄ alkyl, OH, C₁₋₄ alkoxy, or oxo which is formed together with the R^{M′} attached to the same carbon; with the proviso that at least 2 of the R^{M} groups are H; and for each R^{M} which is other than oxo, R^{M′} is H or C₁₋₄ alkyl, with the proviso that when R^{M} is H, then R^{M′} attached to the same carbon is also H.

10. A compound according to claim 9, or a pharmaceutically acceptable salt thereof, wherein:
R² is H, CH₃, CH₂—CH═CH₂, or CH(CH₃)—CH═CH₂;
R³ is H, CH₃, (CH₂)₁₋₃—CH═CH₂, or CH(CH₃)(CH₂)₁₋₂—CH═CH₂;
each R⁴ is independently H or CH₃; and
alternatively, when X is N(R³) and R² and R³ together with the atoms to which they are attached form an azacycloalkyl ring of Formula Ia, then each R^{M} is independently H, CH₃, OH, OCH₃, or oxo which is formed together with the R^{M′} attached to the same carbon; with the proviso that at least 2 of the R^{M} groups are H; and for each R^{M} which is other than oxo, R^{M′} is H.

11. A compound according to claim 1, or a pharmaceutically acceptable salt thereof, which is a compound of Formula II: 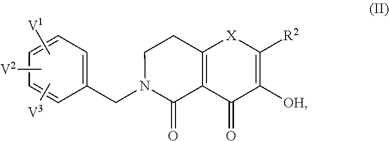
wherein:
X is O or N(R³);
R² is H, C₁₋₄ alkyl, or CH(R^{K})R^{L};
R^{K} is H or C₁₋₄ alkyl;
R^{L} is C(R^{T})═CH₂ or 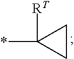
R^{T} is H or C₁₋₄ alkyl;
R³ is H, C₁₋₄ alkyl, or CH(R^{Q})—C₁₋₄ alkenyl;
R^{Q} is H or C₁₋₄ alkyl;
alternatively, when X is N(R³), R² and R³ together with the atoms to which they are attached form an azacycloalkyl ring such that the compound of Formula II is a compound of Formula IIa: 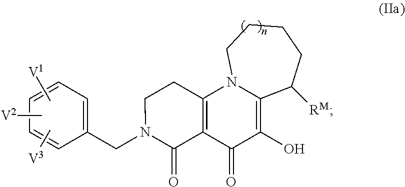
R^{M} is independently H or C₁₋₄ alkyl;
V¹ and V² are each independently:
(1) H,
(2) C₁₋₄ alkyl,
(3) OH,
(4) O—C₁₋₄ alkyl,
(5) C₁₋₄ haloalkyl,
(6) O—C₁₋₄ haloalkyl,
(7) halogen,
(8) CN,
(9) N(R^{A})R^{B},
(10) C(O)N(R^{A})R^{B},
(11) C(O)R^{A},
(12) C(O)OR^{A},
(13) SR^{A},
(14) S(O)R^{A},
(15) SO₂R^{A},
(16) N(R^{A})SO₂R^{B},
(17) N(R^{A})SO₂N(R^{A})R^{B},
(18) N(R^{A})C(O)R^{B},
(19) N(R^{A})C(O)C(O)N(R^{A})R^{B},
(20) HetD,
(21) HetZ, or
(22) C(O)-HetZ, wherein:
HetD is a 5- or 6-membered heteroaromatic ring containing a total of from 1 to 3 heteroatoms independently selected from 1 to 3 N atoms, from zero to 1 O atom, and from zero to 1 S atom, wherein the heteroaromatic ring is optionally substituted with from 1 to 3 substituents each of which is independently C₁₋₄ alkyl, OH, O—C₁₋₄ alkyl, halogen, CN, C(O)N(R^{A})R^{B}, C(O)R^{A}, C(O)OR^{A}, or SO₂R^{A},
HetZ is a 5- or 6-membered saturated heterocyclic ring containing a total of from 1 to 2 heteroatoms selected from 1 to 2 N atoms, zero to 1 O atom, and zero to 1 S atom, wherein the S atom is optionally S(O) or SO₂, wherein the saturated heterocyclic ring is optionally substituted with from 1 to 2 substituents each of which is independently C₁₋₄ alkyl, oxo, C(O)N(R^{A})R^{B}, C(O)R^{A}, CO₂R^{A}, or SO₂R^{A},
and with the proviso that when HetZ is attached to the rest of the compound via the C(O) moiety, then HetZ is attached to the C(O) via a ring N atom;
or alternatively V¹ and V² are respectively located on adjacent carbons in the phenyl ring and together form methylenedioxy or ethylenedioxy;
V³ is:
(1) H,
(2) C₁₋₄ alkyl,
(3) O—C₁₋₄ alkyl,
(4) C₁₋₄ haloalkyl,
(5) O—C₁₋₄ haloalkyl, or
(6) halogen;
each R^{A} is independently H or C₁₋₄ alkyl; and
each R^{B} is independently H or C₁₋₄ alkyl.

12. A compound according to claim 11, or a pharmaceutically acceptable salt thereof, wherein X is N(R³).

13. A compound according to claim 11, or a pharmaceutically acceptable salt thereof, wherein X is O.

14. A compound according to claim 11, or a pharmaceutically acceptable salt thereof, wherein:
R² is H, CH₃, CH₂—CH═CH₂, or CH(CH₃)—CH═CH₂;
R³ is H, CH₃, (CH₂)₁₋₃—CH═CH₂, or CH(CH₃)(CH₂)₁₋₂—CH═CH₂;
R^{M} is H or CH₃.
V¹ and V² are each independently:
(1) H,
(2) CH₃,
(3) CF₃,
(4) OH,
(5) OCH₃,
(6) Cl, Br, or F,
(7) CN,
(8) C(O)NH₂,
(9) C(O)NH(CH₃),
(10) C(O)N(CH₃)₂, or
(11) SO₂CH₃; and
V³ is H, Cl, Br, F, CH₃, or OCH₃.

15. A compound selected from the group consisting of: 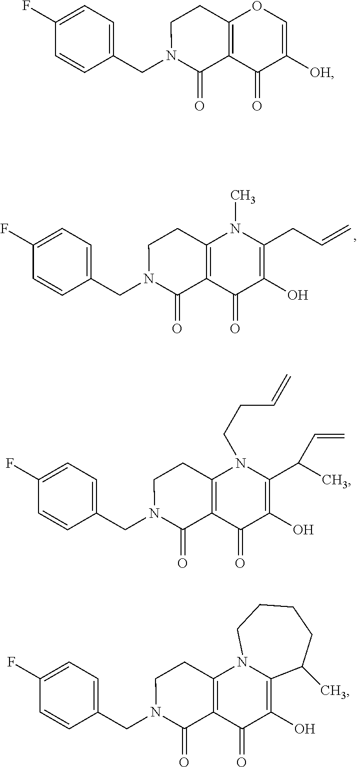
and pharmaceutically acceptable salts thereof.

16. A pharmaceutical composition comprising an effective amount of a compound according to claim 1, or a pharmaceutically acceptable salt thereof, and a pharmaceutically acceptable carrier.

17. (canceled)

18. A method for the treatment or prophylaxis of infection by HIV or for the treatment, prophylaxis, or delay in the onset or progression of AIDS in a subject in need thereof, which comprises administering to the subject an effective amount of the compound according to claim 1, or a pharmaceutically acceptable salt thereof.

19. (canceled)

20. (canceled)
